(19) United States
(12) Patent Application Publication
(10) Pub. No.: US 2026/0355715 A1
(43) Pub. Date: Nov. 12, 2026

(54) TOXIN-BINDER FOR HEMODIALYSIS

(71) Applicants: Universitaet Hamburg, Hamburg (DE); Rheinisch-Westfaelische Technische Hochschule (RWTH) Aachen, Aachen (DE)

(72) Inventors: Tobias Beck, Hamburg (DE); Hendrik Boehler, Heidelberg (DE); Joachim Jankowski, Aachen (DE); Setareh Orth-Alampour, Aachen (DE)

(21) Appl. No.: 19/107,969

(22) PCT Filed: Aug. 29, 2023
(86) PCT No.: PCT/EP2023/073733

§ 371 Date: Feb. 28, 2025

(30) Foreign Application Priority Data
Aug. 31, 2022 (DE) 10 2022 121 980.1

(51) Int. Cl. B01J 20/24; A61M 1/14; B01D 15/08; B01J 20/28

(52) U.S. Cl. CPC B01J 20/24; A61M 1/15632; B01D 15/08; B01J 20/28007; B01J 20/28021; B01J 20/2805

(57) ABSTRACT

New toxin-binders for hemodialysis. An apoferritin nanoparticle for use as a toxin-binder in hemodialysis. The apoferritin nanoparticle comprises apoferritin subunits that, when combined to the apoferritin nanoparticle, form an inner cavity, and the apoferritin nanoparticle does not contain any nanoparticles in its inner cavity. A composition, a sorption cartridge and a dialysis system.

The invention relates to novel toxin binders for hemodialysis.

Approximately 9% of the world's population suffers from chronic kidney disease (CKD) in varying degrees of severity [1]. As the diseases progress, sufferers' vulnerability to cardiovascular diseases increases significantly [2]. Thus, in 2017, 1.2 million people died as a direct consequence of a CKD and a further 1.4 million people died from cardiovascular diseases which were ascribed to a CKD [1]. The high cardiovascular risk has been ascribed to a variety of uremic toxins such as indoxyl sulfate (IS) und para-cresyl sulfate (pCS) [3]-[10]. These toxins in turn can be separated from plasma to only a limited extent with more prolonged or more frequent treatments in the context of the conventional hemodialysis methods [11]. Because of their partially hydrophobic character, the toxins bind to plasma proteins (for example serum albumin), which inhibits efficient diffusion through the pores of the dialysis membranes [12, 13]. These are therefore known as protein-bound uremic toxins (PBUT). On average, 95% of the IS und pCS is protein-bound [14], which explains their low separation efficiency during hemodialysis procedures.

One possibility for improving the separation of PBUTs is the administration of oral or intravenous supplements. Intravenous supplements are intended to force the toxins out of the binding sites of the plasma proteins and therefore increase their soluble fraction and fraction which can be separated by dialysis. This can, for example, be achieved using ibuprofen ((RS)-2-(4-isobutylphenyl) propanoic acid) [15], mesna (sodium-2-sulfanylethane sulfonate) or acetylcysteine [17]. The effective separation of pCS and IS, however, has only been investigated in the case of the administration of ibuprofen. Oral supplements such as the carbon-based AST-120 (Kremezin®) adsorb the toxins generated by microbial processes in the gut prior to uptake into the bloodstream or such as with the synbiotic NatuREN G, inhibit the production of pertinent toxins [19]. In a systematic comparison of all of the studies regarding the reduction of protein-bound uremic toxins up to 02.01.2020 [20], the aforementioned methods exhibited a reduction for the toxins IS and pCS of up to 44% [18]. Significantly higher reductions of between 71 and 78% were, however, obtained with a combination of fractionated plasma separation, adsorption and dialysis (FPAD) [20, 21]. This shows that direct adsorption of the protein-bound toxins is one of the most effective methods for toxin reduction in the plasma of a patient. The disadvantage with this system, however, is the complicated plasma separation and filtration for separating the albumin from the remaining components of the blood, which must not come into contact with the adsorption materials [22], making routine use very expensive.

There is still a lack of effective adsorption materials for PBUTs that have a sufficient biocompatibility and hemocompatibility so that they can be brought into direct contact with the blood and therefore can be integrated more easily into the conventional hemodialysis treatments. Current means for the adsorption of PBUTs do not have a combination of a) higher affinity for the toxins and b) a suitable hemocompatibility and biocompatibility. The prior art only describes investigations into conventional adsorption materials such as, for example, activated carbon [23], metal-organic frameworks or zeolites [25], but the studies were primarily concerned with the affinity of those materials as regards the uremic toxins. Only a few studies also considered the biocompatibility of the materials [23].

DE 10 2016 108 017 A1 describes charged protein containers for the construction of nano-structured materials, wherein the protein containers are constructed from ferritin complexes in the interior of which nanoparticles can be enclosed. Such ferritin complexes containing nanoparticles are also intended, inter alia, for use in dialysis in order to remove hydrophobic toxins. However, no corresponding investigations have been described in this regard. WO 2004/001019 A1 describes nanoparticles which comprise protein cages, for example bacterial ferritin-like protein cages, the interiors of which are loaded with at least one guest material, for example metals.

The objective of the present invention is to provide an adsorbent for separating protein-bound uremic toxins, PBUTs, during dialysis, which has as high an affinity for PBUTs as possible, but has a sufficient biocompatibility and hemocompatibility here to be able to be brought into direct contact with the blood.

The present invention achieves this objective in a first aspect by providing an apoferritin nanoparticle for use as a toxin binder in hemodialysis, wherein the apoferritin nanoparticle comprises apoferritin subunits which form an internal void when assembled into the apoferritin nanoparticle, and wherein the apoferritin nanoparticle does not contain any nanoparticles in its internal void.

Surprisingly, it has been shown that apoferritin nanoparticles are highly suitable for use as adsorbents for toxins in blood, for example for separating protein-bound uremic toxins (PBUTs). The apoferritin nanoparticles in accordance with the invention have an internal void (also termed a cavity below), in which toxins can be bound, and moreover have a high biocompatibility and hemocompatibility. They are therefore highly suitable, in the context of dialysis, for example hemodialysis, for removing toxins, for example PBUTs or in fact other toxins such as heavy metals, from the blood. Thus, for example, they may advantageously be used for the treatment of a chronic kidney disease (CKD) and/or for mitigating the consequences of a chronic kidney disease (CKD). The protein shell of the apoferritin nanoparticle in accordance with the invention has channels or pores through which small molecules such as PBUTs, for example, can pass into the interior of the particle, while larger macromolecules such as proteins or cells are held back. The affinity of the ferritin nanoparticles in accordance with the invention for the toxins to be bound to them can be specifically adjusted and optimized, for example by changing the amino acid sequence of at least one of the subunits in the region of the internal void so that, for example, a suitable number of hydrophobic amino acid residues or a suitable mixture of hydrophobic and hydrophilic amino acid residues is present, or by functionalizing one or more amino acid residues with chemical groups. Furthermore, the transport of material into the ferritin nanoparticles can be improved by enlarging or modifying the surface charge in the region of the pores, for example by specifically exchanging sterically hindering or charged amino acids for smaller hydrophobic amino acids. The inner surface and the region of the pores of the nanoparticle (protein cages) can therefore be varied by sequence modification or chemical functionalization in order to increase the affinity for toxins and/or to facilitate the transport of toxins into the nanoparticles, without compromising the assembly or the biocompatibility. Moreover, the protein cages can also be assembled into a well-defined crystalline material with uniformly distributed solvent channels. This ensures a high purity of the material, facilitates handling and also use as an adsorbent, and is also of assistance in material characterization.

The term “ferritin” (abbreviated to “Ftn”) should be understood to mean a globular protein complex (spheroprotein) which is constructed from 24 protein subunits which form a hollow nanocage and which binds iron in a natural manner. The term “apoferritin” or “apoferritin nanoparticle” refers to the protein complex which is not bound to iron. The protein complex has an internal void approximately 6-8 nm in diameter, while the external diameter is approximately 12-13 nm (see [28], [29], for example). Apoferritin can be composed of identical subunits, for example exclusively from what are known as heavy chains (H-chains), or of varying proportions of a light chain (L-chain) and a heavy chain (H-chain). The H-chain (abbreviated to hFTN-H, HFt or FTH) of human ferritin is encoded by the FTH1 gene and in nature is an approximately 21 kDa long protein comprising 183 amino acids (see UniProtKB No. P02794, 2007 Jan. 23 v2; NCBI NP_002023.2). The light L-chain (abbreviated to hFTN-L, LFt or FTL) of human ferritin is encoded by the FTL gene and in nature is a protein approximately 19.5 kDa long comprising 175 amino acids (see UniProtKB No. P02792, 2007 Jan. 23 v2; NCBI NP_000137.2). Both the light chain and the heavy chain have a tertiary structure with a bundle of four alpha helices (A, B, C and D), wherein the B and C helix are linked together via a non-helical loop consisting of 18 amino acids (BC loop). Furthermore, a fifth alpha helix (E) is present at the C-terminal end and protrudes into the void. The N-terminal end as well as the A and C helices lie on the outside of the ferritin, while the B and D helices are oriented towards the interior of the ferritin. Ferritin has channels or pores in its protein shell which connect the internal void with the external environment ([28]). In this regard, at each site at which three apoferritin subunits contact each other because of the spatial arrangement of the apoferritin subunits, threefold channels (triple axis channels) are formed at sites at which three respective apoferritin subunits contact each other; fourfold channels (quadruple axis channels) are formed at sites at which four respective apoferritin subunits contact one another (see [28], for example). An apoferritin nanoparticle assembled from 24 subunits as a rule comprises 8 threefold channels and 6 fourfold channels (see [28], for example).

The terms “human H-chain ferritin” or “human heavy chain ferritin”, abbreviated to HuHF, refers to a human ferritin the subunits of which consist exclusively of the human heavy chain and not of a mixture of a heavier (H-chain) and a lighter chain (L-chain). The term also encompasses variants with H-chains the amino acid sequence of which has been modified with respect to the wild-type sequence for the H-chain (see UniProtKB No. P02794, 2007 Jan. 23 v2; NCBI NP_002023.2 for the amino acid sequence of the heavy chain of human ferritin).

The term “toxin binder” in respect of the apoferritin nanoparticle means that the apoferritin nanoparticle binds a toxin in its void, i.e. the internal cavity, so that it can be removed with it bound to the apoferritin nanoparticle, for example from a bodily fluid such as blood. Binding between the toxin and the apoferritin nanoparticle in this regard is preferably non-covalent, and may, for example, be formed by hydrophobic interactions, electrostatic interactions or hydrogen bonds. Examples of toxins which may be considered are protein-bound uremic toxins such as indoxyl sulfate (IS) and para-cresyl sulfate (pCS), organic heavy metal compounds, heavy metal ions and other chemical substances and compounds which have properties which enable binding, preferably via non-covalent binding. However, covalent binding is not excluded.

As a rule, the terms “protein-bound uremic toxins” or “PBUTs” refer to chemical compounds which have poor solubility in water (hydrophobic), which are present in serum bound to protein. Examples of PBUTs are indoxyl sulfate (IS), para-cresyl sulfate (p-cresyl sulfate, pCS), phenyl acetate (PheAc, PhAc), p-hydroxyhippuric acid, kynurenine, kynurenic acid, indole-3-acetic acid, 3-carboxy-4-methyl-5-propyl-2-furanpropanoic acid (CMPF) and p-cresylglucuronide (see [37],for example; “Uremic Solutes Database”, “List of uremic solutes”, https://database.uremic-toxins.org/soluteList.php?sortkey=class&direction=asc, retrieved on 25.07.2022, which lists 33 PBUTs).

The term “hemodialysis” should be understood to mean an extracorporeal treatment of the blood, in which the metabolic waste products (for example urea, uric acid, creatinine, excess phosphate, etc.) and/or excess fluid are removed from the blood. Hemodialysis is regularly employed when a person's kidneys no longer function properly, for example in the case of a chronic kidney disease (CKD). The expression “for use in hemodialysis” encompasses use in the context of hemapharesis, in particular whole blood apharesis. The term “hemapharesis” should be understood to mean a method for the extracorporeal removal of specific blood components or of pathogenic substances, microorganisms or the like from blood (see, for example, “Standard der Therapeutischen Apherese [Standards for Therapeutic Apheresis] 2019, 29.03.2019, Deutsche Gesellschaft für Nephrologie).

The term “chronic kidney disease” (CKD) refers to a chronic disease of the kidneys which includes a gradual and long-duration (>3 months) compromise of kidney function up to a permanent kidney insufficiency or total kidney failure (ICD-10-Code N18). The term as used here in particular concerns stage 5 chronic kidney disease (ICD-10-Code N18.5), in which, with a glomerular filtration rate (GFR) of <15 mL/min, a kidney replacement procedure is required in order to clean the blood. The term “chronic kidney insufficiency” as used here is synonymous with the term “chronic kidney disease”.

The expression that the apoferritin nanoparticle contains “no nanoparticles in its internal void” means that the internal void of the apoferritin nanoparticle is empty, i.e. does not include any separate nanoparticles which, for example, are designed for binding toxins.

The term “in the region of the internal void” with reference here to an apoferritin subunit means the portion of the apoferritin subunit, in particular the amino acid residue of this apoferritin subunit, which is oriented towards the internal void of the apoferritin nanoparticle. This is in particular the B helix (in the heavy chain of human apoferritin, amino acids 50-77), the D helix (in the heavy chain of human apoferritin, amino acids 128-159), the E helix (in the heavy chain of human apoferritin, amino acids 165-174) and the C-terminal region following the E helix in the direction of the C-terminal end (175-183).

The term “in the region of a threefold channel” in relation to an apoferritin subunit here means the portion of the apoferritin subunit, in particular the amino acid residues of this apoferritin subunit which, in an assembled apoferritin nanoparticle, is located at a threefold channel of the apoferritin nanoparticle, or amino acid residues which have a direct influence on the transport of a toxin into the interior of the apoferritin nanoparticle, i.e. the exchange of which leads to a facilitation of toxin transport into the interior of the apoferritin nanoparticle. It may, for example, concern amino acids at the end of the C helix and the start of the D helix (in the heavy chain of human apoferritin, for example the amino acids 118 to 141, preferably 120-140). The term also encompasses amino acids on the outside of the apoferritin subunit. “Facilitated toxin transport” (or even “improved toxin transport”) as used here means that upon incubation with a predetermined toxin concentration under suitable conditions within a given time interval, in the case of an apoferritin nanoparticle which is modified in the region of the threefold channel compared with an apoferritin nanoparticle which is not modified in the region of the threefold channel, a greater number of toxin molecules reach the void. The expression “pore” as used occasionally here is synonymous with the term “threefold channel”.

The term “in the region of a fourfold channel” in relation to an apoferritin subunit here means the portion of the apoferritin subunit, in particular the amino acid residues of this apoferritin subunit, which are located at a fourfold channel of the apoferritin nanoparticle. As an example, the amino acids M 158, L 165, Y 168, L 169, H 173 are located in the region of the fourfold channel (see [41], for example). The term encompasses amino acid residues of the respective subunit which, in an assembled apoferritin nanoparticle, are located at a fourfold channel of the apoferritin nanoparticle, for example lining the fourfold channel in an assembled apoferritin nanoparticle or lying at the openings of the channel, or amino acid residues which have a direct influence on the shape, for example the diameter, or the physico-chemical properties of the channel.

The terms “redesigned apoferritin nanoparticle” or “redesigned apoferritin subunit” are used here in connection with an apoferritin nanoparticle or an apoferritin subunit with an amino acid sequence which is modified with respect to the wild-type sequence. The term encompasses an apoferritin nanoparticle or an apoferritin subunit with a modified amino acid sequence in which a portion or all of the exchanged amino acid residues are additionally functionalized with chemical groups.

The term “functionalized” means that an additional chemical group or a compound residue is covalently bound to a suitable amino acid residue, for example cysteine. An example is a cysteine residue, to the thiol group (SH group) of which a N-phenylacetamide residue is bound.

The term “organic compound residue” refers to carbon-containing chemical side groups or molecules that are covalently bound, for example to a cysteine residue. It may be an aliphatic or aromatic compound residue, wherein the terms here should not be understood to be restricted to pure hydrocarbon compounds, but also encompasses heteroaliphatic or heteroaromatic compound residues, i.e. compound residues with heteroatoms such as nitrogen, oxygen, phosphorus or sulfur. The term “aromatic compound residue” should be understood to mean any compound residue which has an aromatic or heteroaromatic group. The term “aliphatic compound residue” should be understood to mean any aliphatic or heteroaliphatic compound residue, i.e. any organic compound residue which does not contain any aromatic groups.

The term “aliphatic compound residue” encompasses cyclic or non-cyclic, linear (straight-chain) or branched, saturated or unsaturated carbon compound residues which are not aromatic residues. The term “heteroaliphatic residue” should be understood to mean aliphatic residues in which one or more C atoms of the carbon framework has been replaced by heteroatoms, for example oxygen, sulfur, nitrogen or phosphorus.

The term “aromatic compound residue” (also designated as “aryl”) refers to chemical groups with aromaticity, including multi-membered aromatic monocyclic groups and polycyclic systems with at least one aromatic ring. Examples of aromatic compound residues (aryl groups) include benzyl and phenyl. The term “heteroaromatic compound residue” (also heteroaryl) should be understood to mean aromatic compound residues in which one or more C atoms of the carbon framework have been replaced by heteroatoms, for example oxygen, sulfur, nitrogen or phosphorus.

The term “dialyzer” refers to an exchangeable blood purification unit of a dialysis system. As an example, a dialyzer may be a hollow fiber membrane module in which biocompatible semi-permeable polymer membranes in the form of hollow fibers are disposed in a housing, so that blood, for example, can flow through the hollow fibers and a dialysate can flow through the interior of the housing on and along the outside of the hollow fibers (for example as a countercurrent to the flow of blood), and preferably, low molecular weight compounds such as salts, protein fragments or nucleic acid fragments can diffuse out of the blood via the membrane into the dialysate and be transported away by it.

A reference to a range being “1-10”, for example, should be understood to mean that every intermediate value is also disclosed. If the reference can only involve integers such as, for example, a number of atoms, this also means that only integers are disclosed. Every narrower range from a broader range is also disclosed by the reference to the broader range, wherein the narrower range also comprises ranges which do not comprise any of the limiting values for the broader range (for example a range of 2-5 out of a range of 1-10).

Amino acids will be referred to here in the form of their three-letter codes or single-letter codes, as appropriate. A list of amino acids with the corresponding codes is provided in the following list:

| Amino acid | Three-letter code | Single-letter code |
|---|---|---|
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

Mutations in protein sequences in the form of amino acid exchanges are given in the format X₁PX₂, wherein X₁ designates the original amino acid (single-letter code) located at a position P and which has been exchanged, X₂ the amino acid (single-letter code) which replaces the original amino acid at the same position following the exchange, and P the position within the amino acid sequence at which the exchange has been made.

The apoferritin nanoparticle for use as a toxin binder during hemodialysis may have an amino acid sequence which is modified with respect to the wild-type sequence in the region of the internal void; for example, it may have cysteine residues at positions at which no cysteine residues are present in the wild-type sequence. The cysteine residues may be further functionalized. As an alternative or in addition, the apoferritin nanoparticle for use as a toxin binder during hemodialysis may have an amino acid sequence which is modified with respect to the wild-type sequence in the region of a threefold channel or a fourfold channel, for example an exchange of amino acids, which results in a facilitated transport of a toxin into the void of the apoferritin nanoparticle.

In a preferred embodiment, at least one of the apoferritin subunits of the apoferritin nanoparticle has an amino acid sequence which is modified with respect to the wild-type sequence in the region of the internal void. In the event that the apoferritin nanoparticle in accordance with the invention is composed of a light (L) and heavy (H) chain with the general structure HₙLₘ, wherein n and m are integers and n+m=24, this means that at least one of the chains, preferably one of the heavy chains, has an amino acid sequence which is modified with respect to the respective wild-type sequence. If the at least one of the apoferritin subunits with a modified amino acid sequence is a subunit from the heavy chain, it is modified with respect to the wild-type sequence for the heavy chain; if the at least one of the apoferritin subunits with a modified amino acid sequence is a subunit from the light chain, it is modified with respect to the wild-type sequence for the light chain. In the case of a human apoferritin nanoparticle, i.e. an apoferritin nanoparticle from the respective human subunits, the pertinent reference is the wild-type sequence for the respective human subunit (see UniProtKB No. P02794, 2007 Jan. 23 v2 or NCBI NP_002023.2 for the heavy chain, UniProtKB No. P02792, 2007 Jan. 23 v2 or NCBI NP_000137.2 for the light chain).

In the present application, the wild-type sequence for the human heavy chain is also given as the sequence with the SEQ ID NO: 1, the wild-type sequence for the human light chain is also given as the sequence with the SEQ ID NO: 2, wherein here, as a distinction from the aforementioned database sequences, the N-terminal methionine has been omitted because it is not a part of the sequence for the completed protein.

SEQ ID NO: 1:
Heavy chain of human ferritin.
TTASTSQVRQNYHQDSEAAINRQINLELYASYVYLSMSYYFDRDDVALKN
FAKYFLHQSHEEREHAEKLMKLQNQRGGRIFLQDIKKPDCDDWESGLNAM
ECALHLEKNVNQSLLELHKLATDKNDPHLCDFIETHYLNEQVKAIKELGD
HVTNLRKMGAPESGLAEYLFDKHTLGDSDNES
SEQ ID NO: 2:
Light chain of human ferritin.
SSQIRQNYSTDVEAAVNSLVNLYLQASYTYLSLGFYFDRDDVALEGVSHF
FRELAEEKREGYERLLKMQNQRGGRALFQDIKKPAEDEWGKTPDAMKAAM
ALEKKLNQALLDLHALGSARTDPHLCDFLETHFLDEEVKLIKKMGDHLTN
LHRLGGPEAGLGEYLFERLTLKHD

In a preferred embodiment of the apoferritin nanoparticle in accordance with the invention for use as a toxin binder in hemodialysis, the amino acid sequence of the at least one apoferritin subunit with an amino acid sequence which is modified with respect to the wild-type sequence is modified compared with the wild-type sequence in the region of the void in a manner such that the apoferritin nanoparticle has a higher binding affinity for a toxin compared with an apoferritin nanoparticle produced from apoferritin subunits the amino acid sequence of which has not been modified with respect to the wild-type sequence. In this embodiment of the apoferritin nanoparticle in accordance with the invention, the modification to the amino acid sequence in at least one of the apoferritin subunits in the region of the internal void is preferably designed in a manner such that the binding affinity of an apoferritin nanoparticle with at least one modified subunit in its amino acid sequence is higher with respect to a toxin to be removed from blood than in the case of an apoferritin nanoparticle with subunits the amino acid sequence of which is unmodified with respect to the wild-type sequence. As an example, the modification may be an increase in the number of hydrophobic amino acid residues. Examples of amino acids with a hydrophobic amino acid residue are alanine, valine, methionine, leucine, isoleucine, proline, tryptophan and phenylalanine. In order to bind specific toxins, however, it may also be advantageous to provide a higher proportion of polar neutral, acidic or basic amino acids in the at least one apoferritin subunit. Examples of amino acids with polar/neutral amino acid residues are tyrosine, threonine, glutamine, glycine, serine, cysteine and asparagine. Examples of acidic amino acids are glutamic acid and aspartic acid. Examples of basic amino acids are lysine or arginine. In order to bind specific toxins, it may also be advantageous to provide a specific mixture and/or distribution of amino acids with hydrophobic and polar/neutral amino acid residues. The binding affinity as regards toxins, for example PBUTs, for apoferritin nanoparticles modified in this manner with respect to unmodified apoferritin nanoparticles can readily be determined by the person skilled in the art.

In a preferred embodiment of the apoferritin nanoparticle in accordance with the invention for use as a toxin binder in hemodialysis, the at least one apoferritin subunit with an amino acid sequence which is modified with respect to the wild-type sequence in the region of the internal void has at least one additional cysteine residue or a cysteine residue in a different position in the sequence compared with the wild-type sequence, wherein the at least one apoferritin subunit with an amino acid sequence which is modified with respect to the wild-type sequence is functionalized or can be functionalized by covalent binding of an organic compound residue, for example an aliphatic or aromatic compound residue, to the cysteine residue. A functionalization preferably serves to increase the binding affinity of the apoferritin nanoparticle in accordance with the invention in respect of toxins to be removed from blood, for example protein-bound uremic toxins.

Particularly preferably, in the case of the at least one apoferritin subunit with an amino acid sequence which is modified with respect to the wild-type sequence, possible cysteine residues which are naturally present in the region of the internal void are replaced by other amino acid residues, for example by alanine residues. This enables specific positioning of the cysteine residues to be introduced and therefore of the sites at which the apoferritin subunit in accordance with the invention can be functionalized. Preferably, in the case of the at least one apoferritin subunit with an amino acid sequence which is modified with respect to the wild-type sequence, then, possible cysteine residues present in the region of the internal void are replaced by another amino acid residue, for example an alanine residue, and at least one cysteine residue is introduced into a position in the region of the internal void at which no cysteine residue is present in the native protein.

Particularly preferably, the at least one apoferritin subunit with an amino acid sequence which is modified with respect to the wild-type sequence in the apoferritin nanoparticle in accordance with the invention for use as a toxin binder in hemodialysis has, compared with the wild-type sequence, two, three or four cysteine residues in the region of the internal void at which a respective organic compound residue is covalently bound or can be bound. Preferably in this embodiment, in the region of the internal void, three or four cysteine residues are present which are each functionalized or can be functionalized in an identical or different manner. In the case in which all 24 subunits of an apoferritin nanoparticle in accordance with the invention is functionalized or can be functionalized in this manner, this results in a number of 72-96 functionalization sites, i.e. sites within the apoferritin nanoparticle which can be functionalized or are functionalized. As described above, preferably, possible cysteine residues of the native protein in the region of the internal void are respectively replaced, for example by alanine residues, and at least one of the introduced cysteine residues is introduced at a position at which no cysteine residue is present in the native protein. The functionalization sites present in an apoferritin nanoparticle in accordance with the invention may be functionalized in an identical or different manner, i.e. respectively functionalized with the same or different chemical groups.

The organic compound residue may be a hydrophobic, hydrophilic or an amphiphilic compound residue. Hydrophobic compound residues are preferred for binding hydrophobic uremic toxins, for example. However, in order to bind hydrophobic uremic toxins, it may be advantageous to provide an amphiphilic compound residue for the functionalization, or to functionalize different cysteine residues in the region of the internal void with different compound residues, for example with a mixture of hydrophilic and hydrophobic compound residues. The adjustment of the hydrophobicity or hydrophilicity of a compound residue is known to the person skilled in the art and can if necessary be determined by routine experiments. As an example, the hydrophobicity of a compound residue may be influenced by the size or length of a hydrocarbon residue, and the hydrophilicity by the presence of polar or charged residues.

The organic compound residue may, for example, be an acetamidyl compound with the general structure —CH₂—C(O)—NH—R. The residue R may be an aliphatic, heteroaliphatic, aromatic or heteroaromatic residue. In the case of aliphatic and heteroaliphatic residues, a maximum chain length, i.e. a maximum number of component atoms of the compound residue, of 35 atoms is preferred, particularly preferably a maximum chain length of 34, 33, 32, 31 or 30 atoms. In the case of aromatic and heteroaromatic residues, electron-depleted aromatic systems are preferred which allow effective π-π interactions with electron-rich aromatic systems of the toxins. Examples of electron-depleted aromatic residues are 3,4,5-trinitrophenyl, 3,4,5-trihalogenyl (for example 3,4,5-trifluorophenyl) or 3,4,5-tris(trihalogenomethyl) phenyl residues (for example 3,4,5-tris(trifluoromethyl) phenyl residues). Examples of electron-depleted heteroaromatic groups are pyridine, quinoline or isoquinoline, the electron-depleted character of which can be further strengthened with electron-attracting substituents such as nitro groups, tertiary amine groups, positively charged groups or halogen atoms. In the case of aromatic and heteroaromatic residues, a maximum molecular size of 45, preferably a maximum of 44, 43, 42, 41 or 40 atoms is preferable, i.e. residues which are composed of a maximum of 45, preferably a maximum of 44, 43, 42, 41 or 40 atoms, including heteroatoms. In the case of aromatic and heteroaromatic residues, a maximum molecular size of 39 atoms, particularly preferably a maximum of 38, 37, 36, 35, 34, 33, 32, 31 or 30 atoms, is more preferable. Because the overwhelming proportion of PBUTs have a formal negative charge under physiological conditions, amphiphilic compound residues with a combination of hydrophobic groups with positively charged groups are preferred. Examples of groups which are positively charged under physiological conditions are primary, secondary, tertiary and quaternary amines. In the case of heteroaliphatic compounds, it is preferable for these groups to be contained in the chain. Depending on the geometry of the toxin to be bound, these groups could also be integrated into the chain in alternation, wherein preferably, a maximum chain length of 35 atoms, particularly preferably 34, 33, 32, 31 or 30 atoms, is not exceeded. In the case of aromatic and heteroaromatic residues, the charged groups are preferably disposed in suitable symmetry with respect to the aromatic or heteroaromatic system. Here again, it is preferable that a maximum molecular size of the compound residue is not exceeded, preferably a molecular size of 45, preferably 44, 43, 42, 41 or 40 atoms; more preferably, a molecular size of 39 atoms, particularly preferably 38, 37, 36, 35, 34, 33, 32, 31 or 30 atoms is not exceeded. For toxins such as p-cresyl sulfate or phenylacetic acid, 1-amino-2-phenylethyl residues or 1,1-diamino-2-phenylethyl residues, for example, are suitable. Preferred compound residues for binding protein-bound uremic toxins which can be covalently bound or are bound covalently to the at least one additional cysteine residue or a cysteine residue disposed at another position in the sequence are selected from the group consisting of N-phenylacetamidyl, N-decylacetamidyl, N-(1-amino-2-phenylethyl) acetamidyl, N-(1-amino-2-phenylpropyl) acetamidyl, N-(1-amino-2-phenylbutyl) acetamidyl, N-(1,1-diamino-2-phenylethyl) acetamidyl, N-(1,1-diamino-2-phenylpropyl) acetamidyl, N-(1,1-diamino-2-phenylbutyl) acetamidyl, N-(3,4,5-tris(trifluoromethyl)phenyl) acetamidyl, N-(3,4,5-trinitrophenyl) acetamidyl, N-(1-amino-2-(3,4,5-trinitrophenyl)ethyl) acetamidyl and N-(2-(heptylamino)ethyl) acetamidyl. The cited compounds are preferably covalently bound to the cysteine residue via the acetamide group. For this purpose, halogenated acetamide derivatives may be used, which react with the thiol group of the cysteine. Furthermore, clearly it is also possible to use other suitable coupling groups, for example haloalkyl or maleimide derivatives, for binding to the thiol group.

The organic compound residue may also, for example, be a chelating agent which binds heavy metals such as lead, antimony, arsenic, cadmium, nickel, mercury, thallium or uranium, for example. Examples of chelating agents (complex-forming agents) are ethylenediaminetetraacetic acid (EDTA), dimercaptosuccinic acid (DMSA) or dimercaptopropanesulfonic acid (DMPS). The term “heavy metal” encompasses heavy metal ions.

In a preferred embodiment of the apoferritin nanoparticle in accordance with the invention for use as a toxin binder in hemodialysis, the amino acid sequence of the at least one apoferritin subunit with an amino acid sequence which is modified with respect to the wild-type sequence—as an alternative or in addition to a modification to the amino acid sequence in the region of the internal void—may be modified compared with the wild-type sequence in the region of a threefold channel in a manner such that the transport of a toxin into the void of the apoferritin nanoparticle through the threefold channel is facilitated compared with an apoferritin nanoparticle with an unmodified amino acid sequence. In this embodiment, the amino acid sequence of at least one apoferritin subunit may be modified with respect to the wild-type sequence only in the region of a threefold channel, for example in order to facilitate only the transport into the apoferritin nanoparticle, without modifications to the amino acid sequence in the region of the internal void also being present, or the amino acid sequence of at least one apoferritin subunit may also be modified with respect to the wild-type sequence in the region of a threefold channel, in addition to a modification to the amino acid sequence in the region of the internal void. Preferably, at least one modification to the amino acid sequence is present in the region of the threefold channel in at least two, preferably three apoferritin subunits forming a threefold channel. As an example, respectively one, two, three or more, for example up to 12 amino acid modifications may be present in one, two or three of the apoferritin subunits forming a threefold channel. In the case of modifications to two or three of the apoferritin subunits, these may be identical or different for each subunit, but preferably, they are identical. An apoferritin nanoparticle for use as a toxin binder in hemodialysis which is composed of identical subunits which each have identical modifications to the amino acid sequence with respect to the wild-type sequence in the region of a threefold channel, for example at least at one, two, three, four, five, six, seven, eight, nine, ten, eleven or twelve positions of amino acids in the region of the threefold channel, is particularly preferred. As already mentioned, these modifications may be present alone or also in combination with a modification to the amino acids in the region of the internal void.

In the context of hemodialysis with the aid of the apoferritin nanoparticle in accordance with the invention, for binding to a toxin, this is preferably a protein-bound uremic toxin which is preferably selected from the group consisting of indoxyl sulfate, para-cresyl sulfate, phenyl acetate and p-hydroxyhippuric acid.

The apoferritin nanoparticle in accordance with the invention for use as a toxin binder in hemodialysis preferably has an amino acid sequence which is modified with respect to the wild-type sequence in the region of the internal void and/or in the region of a threefold channel and/or in the region of a fourfold channel in at least two, more preferably at least three, at least four, at least five, at least six, at least seven, at least eight, at least nine or at least 10, more preferably at least 12, at least 14, at least 16, at least 18, at least 20 or at least 22, particularly preferably all 24 subunits. Preferably, the subunits from which the apoferritin nanoparticle in accordance with the invention is composed are identical having regard to their amino acid sequence. More preferably, in accordance with one embodiment, an apoferritin nanoparticle in accordance with the invention as described above is modified by the introduction of cysteine residues which are functionalized with suitable chemical compound residues, wherein preferably, at least two, at least three, at least four, at least five, at least six, at least seven, at least eight, at least nine or at least 10, more preferably at least 12, at least 14, at least 16, at least 18, at least 20 or at least 22, preferably all 24 subunits are modified and functionalized in the same manner. In one embodiment in which the apoferritin nanoparticle has a modified amino acid sequence in the region of a threefold channel, be it alone or in combination with a modified amino acid sequence in the region of the internal void, preferably has at least two, more preferably at least three, at least four, at least five, at least six, at least seven, at least eight, at least nine or at least 10, more preferably at least 12, at least 14, at least 16, at least 18, at least 20 or at least 22, preferably all 24 subunits have an amino acid sequence which is modified with respect to the wild-type sequence in the region of a threefold channel.

In a preferred embodiment of an apoferritin nanoparticle in accordance with the invention for use as a toxin binder in hemodialysis, preferably at least two, more preferably at least three, at least four, at least five, at least six, at least seven, at least eight, at least nine or at least 10, more preferably at least 12, at least 14, at least 16, at least 18, at least 20 or at least 22, preferably all 24 subunits have an amino acid sequence which is modified with respect to the wild-type sequence in the region of the internal void and/or in the region of a threefold channel and/or in the region of a fourfold channel which is selected from one of the amino acid sequences in accordance with SEQ ID NO: 13-24. In this regard, preferably, the subunits with an amino acid sequence which is modified with respect to the wild-type sequence each have the same sequence. If, for example, an apoferritin nanoparticle in accordance with the invention for use as a toxin binder in hemodialysis has 24 subunits with an amino acid sequence which is modified with respect to the wild-type sequence in the region of the internal void and/or in the region of a threefold channel and/or in the region of a fourfold channel, preferably, the 24 subunits have the same amino acid sequence, for example the amino acid sequence in accordance with SEQ ID NO: 13, SEQ ID NO: 17 or SEQ ID NO: 24. Preferably, the subunits have an amino acid sequence which is selected from one of the amino acid sequences in accordance with SEQ ID NO: 17, SEQ ID NO: 18, SEQ ID NO: 23, or SEQ ID NO: 24.

Particularly preferably, the apoferritin nanoparticle in accordance with the invention is exclusively composed of subunits consisting of the heavy chain, particularly preferably the human heavy chain. As regards the amino acid sequence, the chain may also be modified with respect to the wild-type sequence at the protein regions lying on the outside (for example the A and C helices), for example in order to produce a specific external charge, for example a negative or positive charge. Furthermore, particularly preferably, the apoferritin nanoparticle in accordance with the invention does not have any cysteine residues on the outside of the nanoparticle but, if appropriate, exclusively in the internal void, so that a functionalization with an organic compound residue exclusively occurs at the cysteine residues in the internal void. A functionalization may be obtained by disassembling the apoferritin nanoparticle under suitable conditions into its subunits and then functionalizing the individual subunits. After reestablishing the original conditions, the subunits then spontaneously re-form the apoferritin nanoparticle. Suitable conditions are known to the person skilled in the art and are described, for example, in [31], [38], and also below.

In a second aspect, the invention also concerns a composition for use as a toxin binder in hemodialysis, comprising a plurality of apoferritin nanoparticles in accordance with the invention for use as a toxin binder in hemodialysis in accordance with the first aspect of the invention described above. A composition of this type may advantageously be used as an adsorbent for binding toxins in blood, for example for binding protein-bound uremic toxins (PBUTs) or for binding heavy metals.

In the composition in accordance with the invention for use as a toxin binder in hemodialysis, the plurality of apoferritin nanoparticles in accordance with the invention for use as a toxin binder in hemodialysis may be in the amorphous form or in a defined arrangement, for example in a crystalline form. The term “composition of apoferritin nanoparticles in an amorphous form” should be understood here to mean a composition of apoferritin nanoparticles in which the apoferritin nanoparticles are linked together with only the formation of a disordered or indistinct, in particular non-crystalline structure. A defined, for example crystalline, arrangement of apoferritin nanoparticles in accordance with the invention may be obtained in a manner which is known to the person skilled in the art (see [31], for example). In the case of an arrangement in a crystalline form, for example, the apoferritin nanoparticles for use as a toxin binder in hemodialysis contained in the arrangement are preferably crosslinked. An adsorbent of this type has apoferritin nanoparticles in accordance with the invention as protein cages, which are assembled into a defined crystalline material with uniformly distributed solvent channels. This is not only advantageous in obtaining a high purity for the material, but is also advantageous for handling when used as a sorbent material. As an example, the apoferritin nanoparticles which have been crosslinked into a crystalline structure can be handled as a whole and separate from the blood, for example. The form of the crystal may, for example, be adapted to the form of the void of a sorbent cartridge. In the composition in accordance with the invention, the plurality of apoferritin nanoparticles in accordance with the invention for use as a toxin binder in hemodialysis may, for example, be present in water or an aqueous solution, for example a suitable aqueous buffer solution. The aqueous solution is preferably a biocompatible or pharmaceutically acceptable solution in which the nanoparticles are stable.

In a third aspect, the present invention also concerns a sorbent cartridge which comprises a plurality of apoferritin nanoparticles in its interior for use as a toxin binder in hemodialysis in accordance with the first aspect of the invention or a composition for use as a toxin binder in hemodialysis in accordance with the second aspect of the invention. A sorbent cartridge in accordance with the invention may, for example, be constructed such that it can be integrated into a dialysis system. To this end, the sorbent cartridge preferably has an inlet for the supply of blood and an outlet for blood to leave.

Preferably, the sorbent cartridge is constructed in a manner such that the plurality of apoferritin nanoparticles or the composition comprising the plurality of apoferritin nanoparticles can be retained in the sorbent cartridge by suitable means in the sorbent cartridge. As an example, the plurality of apoferritin nanoparticles or the composition may be contained in a first compartment inside the sorbent cartridge which is separated from a second compartment inside the sorbent cartridge by one or more membranes, wherein the membrane or the membranes are permeable to unbound toxin molecules and/or to proteins with toxin molecules bound thereto, but not permeable to the apoferritin nanoparticles. Blood which is to be purified of toxin molecules, for example PBUTs, may, for example, be conducted through the second compartment. Toxin molecules present in the blood, in the free form or, if appropriate, also bound to proteins, can reach the first compartment via the membrane(s), for example by diffusion, and be bound there by the apoferritin nanoparticles in accordance with the invention. In a particularly preferred embodiment of the sorbent cartridge in accordance with the invention, the plurality of apoferritin nanoparticles or the composition are, however, disposed in the sorbent cartridge in a manner such that in operation, for example in a dialysis system, direct contact occurs with the blood to be purified. In this preferred embodiment, a crystalline or non-crystalline (amorphous) composition of the apoferritin nanoparticles is located inside the sorbent cartridge or at least inside a compartment inside the sorbent cartridge, wherein blood can come into direct contact with the composition inside the sorbent cartridge. In this form, the sorbent material in accordance with the invention forms an insoluble (amorphous or crystalline) solid which is significantly larger than the components of the blood (for example erythrocytes, at ca. 2.5 μm). The difference in size makes separation easy. The sorbent material can be retained using relatively coarse filters, while the blood flows through the sorbent cartridge. Here, the biocompatibility of the material in accordance with the invention proves to be particularly advantageous, because it is not necessary to separate the sorbent material from the blood, whereupon not only is closer contact between the sorbent material and the toxins to be separated made possible, but also, integration into existing dialysis systems is facilitated.

In a fourth aspect, the invention concerns a dialysis system comprising a dialyzer and at least one sorbent cartridge in accordance with the third aspect of the invention.

The at least one sorbent cartridge may be disposed a) upstream, b) downstream in the direction of flow of the blood to be dialyzed, or c) in an auxiliary circuit to the dialysis circuit with respect to the dialyzer.

In a fifth aspect, the invention also concerns a method for the dialysis of blood, preferably human blood, comprising the step of bringing the blood into contact with an apoferritin nanoparticle in accordance with the invention in accordance with the first aspect of the invention or with a composition in accordance with the second aspect of the invention. The step of contacting is preferably carried out over a time and under conditions which are sufficient to bind a toxin contained in the blood, for example a PBUT or a heavy metal, to the apoferritin nanoparticles or to the composition. The composition may comprise the apoferritin nanoparticles in an amorphous or crosslinked (crystalline) form. The term “contacting” as used here in particular encompasses bringing blood into direct contact with the apoferritin nanoparticles or the composition, i.e. without separation by a semipermeable membrane, for example. The dialysis method in accordance with the invention is particularly advantageous for the treatment of a chronic kidney disease (CKD) and/or for mitigating the consequences of a chronic kidney disease (CKD).

In a further aspect, the invention therefore also concerns a method for the therapeutic treatment of a patient with a chronic kidney disease (CKD) and/or for the therapeutic mitigation of the consequences of a chronic kidney disease (CKD), comprising a method for the dialysis of blood in accordance with the above fifth aspect of the invention.

The invention will now be described in more detail purely by way of illustration, with the aid of the accompanying drawings and exemplary embodiments.

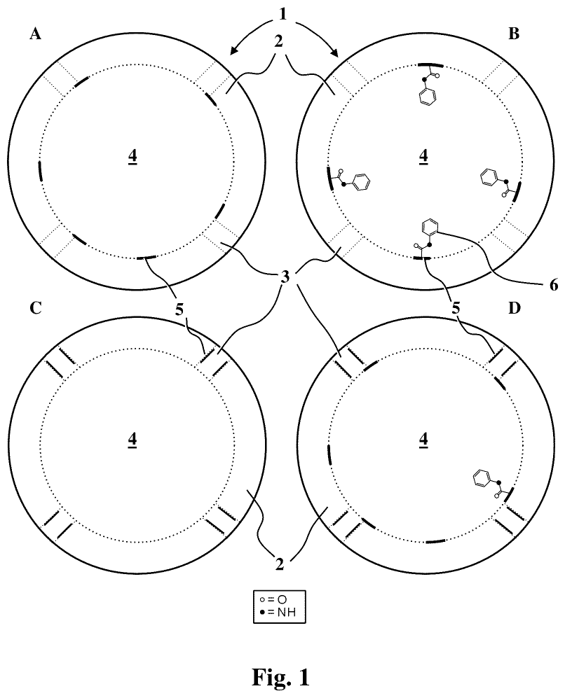

FIG. 1. Schematic representation of embodiments of the apoferritin nanoparticles in accordance with the invention. A. Schematic representation of an apoferritin nanoparticle in accordance with the invention with modification to the amino acid sequence in the region of the internal void; B. Schematic representation of an apoferritin nanoparticle with modification to the amino acid sequence in the region of the internal void with functionalized cysteine residues; C. Schematic representation of an apoferritin nanoparticle with modification to the amino acid sequence in the region of a threefold channel; D. Schematic representation of an apoferritin nanoparticle with modification to the amino acid sequence in the region of the internal void (with functionalization of a cysteine residue), and with additional modification to the amino acid sequence in the region of a threefold channel.

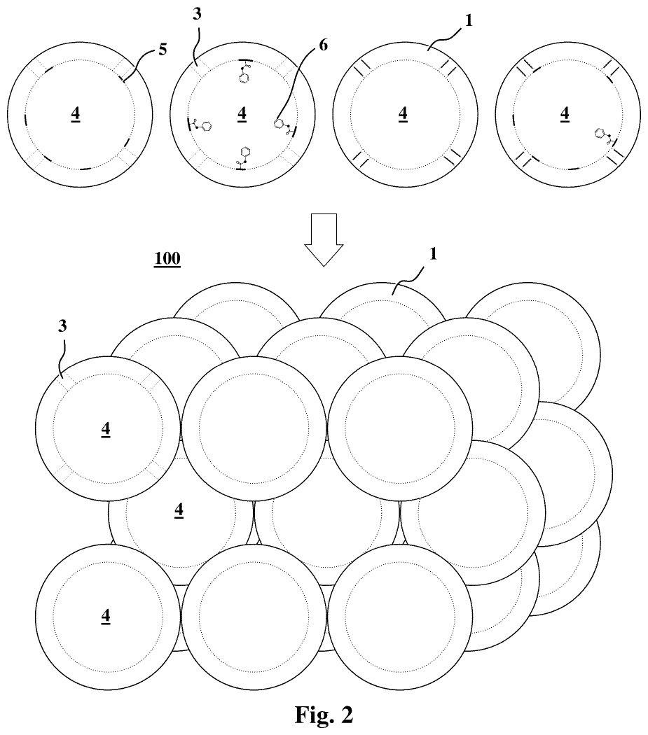

FIG. 2. Schematic representation a crystal-type arrangement (lower portion of the figure) of a plurality of apoferritin nanoparticles in accordance with the invention, assembled from apoferritin nanoparticles from FIG. 1 (upper portion of the figure).

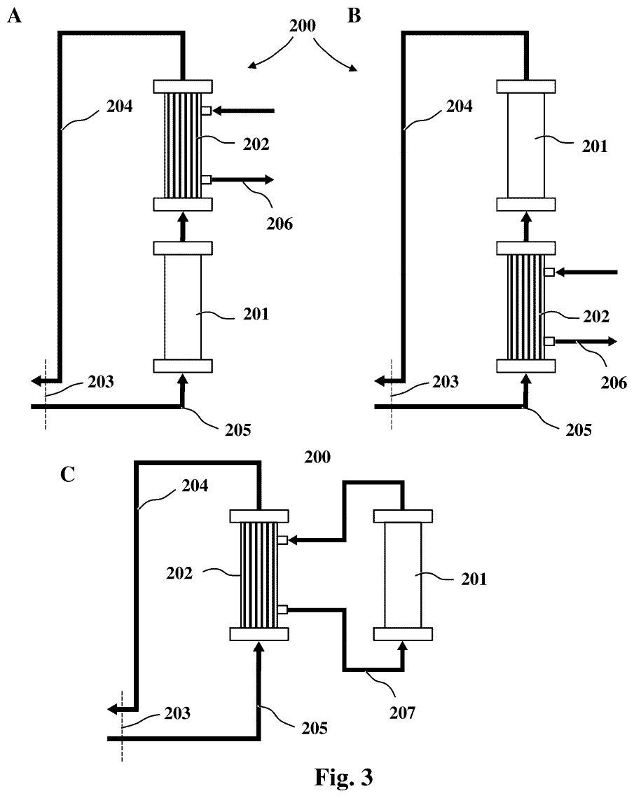

FIG. 3. Schematic representation of embodiments of a dialysis system in accordance with the invention. A. Dialysis system with upstream sorbent cartridge in accordance with the invention; B. Dialysis system with downstream sorbent cartridge in accordance with the invention; C. Dialysis system with sorbent cartridge in accordance with the invention in an auxiliary circuit.

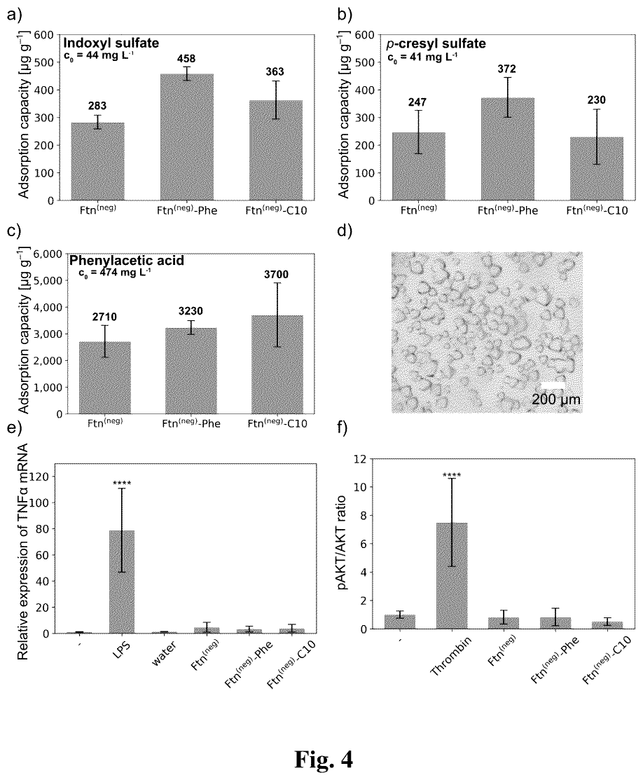

FIG. 4. Results of adsorption experiments with functionalized apoferritin nanoparticles for binding PBUTs. a), b), c): Adsorption capacity of untreated and functionalized ferritin with respect to representative PBUTs in concentrations which are expected in CKD patients' blood [26]. d) Optical microscopic image of the crystals of Ftn-Phe acting as an adsorbent. e) Ratio of phosphorylated AKT to AKT as a measure of the activation of blood platelets and associated blood coagulation f) Relative concentration of TNF-α mRNA in endothelial cells. Ftn^{(neg)}-Phe: Ftn^{(neg)}-apoferritin nanoparticle functionalized with 2-iodo-N-phenylacetamide (Phe); Ftn^{(neg)}-C10: Ftn^{(neg)}-apoferritin nanoparticle functionalized with 2-bromo-N-decylacetamide (C10).

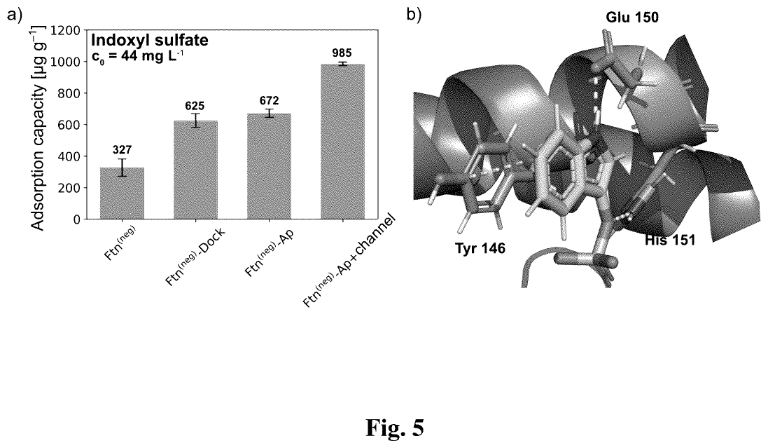

FIG. 5. a) Adsorption capacity of genetically modified ferritin variants in respect of indoxyl sulfate. Ftn^{(neg)}-Dock: Variant with toxin binding site (sequence: Ftn^{(neg)}-dock03, SEQ ID NO: 21); Ftn^{(neg)}-Ap: Variant with reduced negative surface charge on the internal surface (sequence: Ftn^{(neg)}-Ap4, SEQ ID NO: 17); Ftn^{(neg)}-Ap-channel: Variant with reduced negative surface charge on the internal surface and in the region of the threefold channel (sequence: Ftn^{(neg)}-Ap4-3A, SEQ ID NO: 18). b) Indoxyl sulfate and its interactions with three side chains in a designed toxin binding site (computer model).

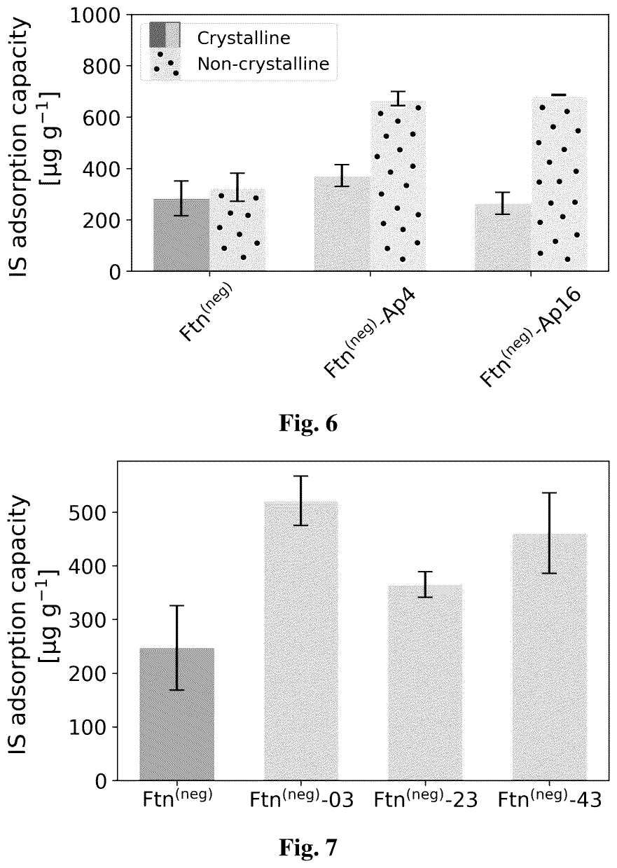

FIG. 6. Adsorption capacity of crystalline (left) and non-crystalline (right) genetically modified ferritin variants in accordance with the invention in respect of indoxyl sulfate (IS). Ftn (neg): apoferritin nanoparticle variant with increased negative charge on the external surface (SEQ ID NO: 12); Ftn^{(neg)}-Ap4: variant with 4 mutations compared with Ftn (neg) and accordingly modified reduced negative surface charge on the internal surface (SEQ ID NO: 17); Ftn^{(neg)}-Ap16: variant with 16 mutations with respect to Ftn (neg) and accordingly modified reduced negative surface charge on the internal surface (SEQ ID NO: 20).

FIG. 7. Adsorption capacity of crystalline genetically modified ferritin variants in accordance with the invention with specially developed binding sites in respect of indoxyl sulfate (IS) compared with Ftn (neg). Ftn^{(neg)}-03 (=Ftn^{(neg)}-dock03, SEQ ID NO: 21); Ftn^{(neg)}-23 (=Ftn^{(neg)}-dock23, SEQ ID NO: 22); Ftn^{(neg)}-43 (=Ftn^{(neg)}-dock43, SEQ ID NO: 22).

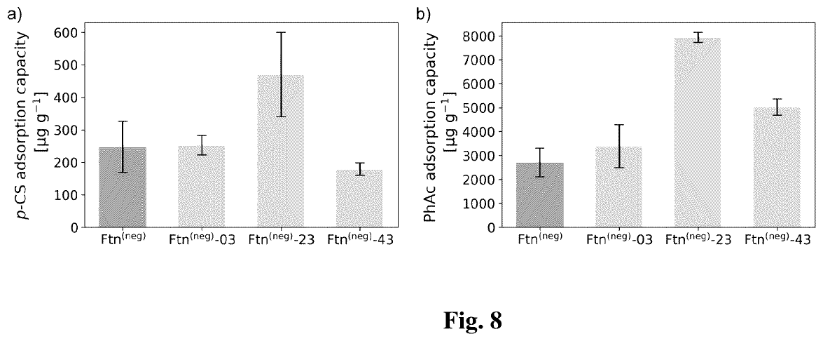

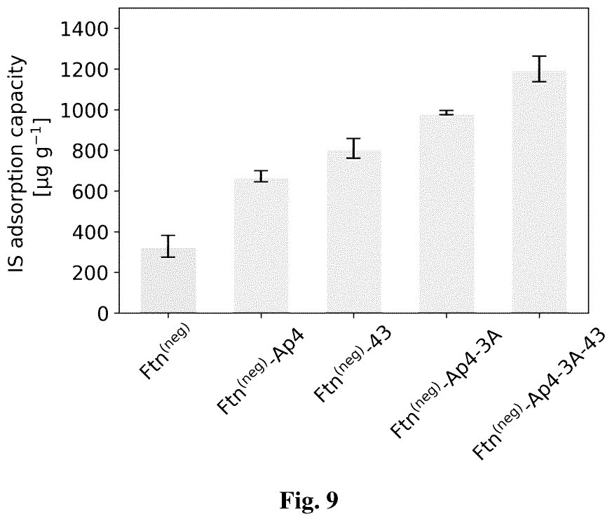

FIG. 8. Adsorption capacity of crystalline genetically modified ferritin variants in accordance with the invention with specially developed binding sites compared with para-cresyl sulfate (pCS) and phenyl acetate (PhAc) compared with Ftn (neg). Ftn^{(neg)}-03 (=Ftn^{(neg)}-dock03, SEQ ID NO: 21); Ftn^{(neg)}-23 (=Ftn^{(neg)}-dock23, SEQ ID NO: 22); Ftn^{(neg)}-43 (=Ftn^{(neg)}-dock43, SEQ ID NO: 22).

FIG. 9. Adsorption capacity of a crystalline genetically modified ferritin variant in accordance with the invention (Ftn^{(neg)}-Ap4-3A-dock43; SEQ ID NO: 24) with combined sequence modifications in respect of indoxyl sulfate (IS) compared with Ftn^{(neg)} and the variants Ftn^{(neg)}-Ap4 (SEQ ID NO: 17), Ftn^{(neg)}-Ap4-3A (SEQ ID NO: 18) and Ftn^{(neg)}-dock43 (SEQ ID NO: 23), Ftn^{(neg)}-43=Ftn^{(neg)}-dock43; Ftn^{(neg)}-Ap4-3A-43=Ftn^{(neg)}-Ap4-3A-dock43.

FIG. 1 shows a greatly simplified and schematic representation of embodiments of apoferritin nanoparticle 1 in accordance with the invention for use as a toxin binder in hemodialysis. As can be seen from FIG. 1, the apoferritin nanoparticle 1 has a generally spherical shape with a protein shell formed from apoferritin subunits 2 which encloses an internal void 4, and in which channels 3 are formed at the interfaces between the apoferritin subunits 2, through which toxins can pass from the external environment, for example by diffusion, into the internal void 4, and be bound therein, for example by hydrophobic interactions. In FIG. 1, four individual apoferritin nanoparticles 1 are shown schematically, wherein in FIG. 1A, an embodiment is shown in which, in the region of the internal void 4, sequence modification sites 5 are disposed, i.e. sites at which modifications to the amino acid sequence of the apoferritin subunits 2 have been made, i.e. individual amino acids or whole amino acid sequence regions with respect to the wild-type sequence for the respective apoferritin subunit 2 have been exchanged for other amino acids or amino acid sequence regions. A functionalization of the amino acids, for example cysteine residues, was not carried out here. Binding of toxins can occur, for example, by hydrophobic interaction with hydrophobic amino acid residues present at the sequence modification sites 5. In the embodiment of an apoferritin nanoparticle 1 for use as a toxin binder in hemodialysis shown in FIG. 1B, firstly, possible native cysteine residues which were present have been replaced by alanine and then at four sequence modification sites 5, four amino acids have been replaced by cysteine which had been functionalized at its SH group with an organic compound residue covalently bound thereto (here, a N-phenylacetamidyl residue, for example). In the example, four cysteine residues have been functionalized with one hydrophobic organic compound residue in order to bind uremic toxins. FIG. 1C schematically shows an embodiment of an apoferritin nanoparticle 1 for use as a toxin binder in hemodialysis, in which only sequence modification sites 5 in the region of a threefold channel have been provided, i.e. an amino acid sequence which has been modified with respect to the wild-type sequence is only present in the region of a threefold channel. FIG. 1C schematically shows an embodiment of an apoferritin nanoparticle 1 for use as a toxin binder in hemodialysis in which, in addition to sequence modification sites 5 in the region of a threefold channel, sequence modification sites 5 are also present in the region of the internal void 4, wherein here, by way of example, one of the sequence modification sites 5 is shown as a functionalized sequence modification site 5.

FIG. 2 schematically shows the assembly of apoferritin nanoparticles 1 in accordance with the invention (upper part of the figure) into a crystal-like composition 100 (lower part of the figure). Under suitable conditions, the apoferritin nanoparticles 1 can be assembled into a well-defined macroscopic crystalline material with uniformly distributed solvent channels. This guarantees high purity for the material as well as easy handling, and is also helpful as regards material characterization. In order to fix the arrangement, the apoferritin nanoparticles 1 may be crosslinked among one another, if appropriate.

FIG. 3 schematically shows different embodiments of a dialysis system 200 in accordance with the invention. FIG. 2A shows an embodiment in which a sorbent cartridge 201 is connected upstream of a dialyzer 202, in this case a hollow fiber membrane module, in the direction of flow of blood given by the arrow. The flow direction is determined by an appropriate pump, which is not shown here. The body periphery is schematically shown here by the dashed line 203. Blood is conducted to the sorbent cartridge 201 via a line 205, for example a tube. An adsorbent in accordance with the invention, for example a composition 100 in the form of a crystalline arrangement in accordance with the second aspect of the invention, is located in the sorbent cartridge 201. The blood can be brought into direct contact with the composition 100 containing the apoferritin nanoparticles 1 in accordance with the invention. Possible uremic toxins contained in the blood, for example uremic toxins bound to proteins, can be bound by the adsorbent. The blood which has been freed from toxins or at least depleted in toxins is returned to the dialyzer 202 and then back into the patient's body via a line 204 leading to the body. A dialysate circuit 206 may serve for exchange of or as a countercurrent of dialysate.

FIG. 3B shows a dialysis system 200 in accordance with the invention, in which the sorbent cartridge 201 in accordance with the invention is downstream of the dialyzer 202 in the direction of blood flow. In this embodiment, the dialysis system 200 otherwise corresponds to that of FIG. 3A, and so reference should be made to the discussion above for further details. Here again, the blood to be freed of or depleted in toxins comes into direct contact with the apoferritin nanoparticles 1 or a composition 100 comprising the apoferritin nanoparticles 1 in the sorbent cartridge 201.

FIG. 3C shows a dialysis system 200 in accordance with the invention, in which the sorbent cartridge 201 in accordance with the invention with apoferritin nanoparticles 1 in accordance with the invention is disposed in an auxiliary circuit 207 to the blood circulation. Instead of or in addition to a dialysate circuit, not shown here, adsorbent can be conducted into the dialyzer 202 in order to bind toxins from the blood passing over the hollow fibers via the membranes. In this embodiment, the blood to be purified does not come into direct contact with the apoferritin nanoparticles 1 or a composition 100 comprising the apoferritin nanoparticles 1.

### EXAMPLES

### 1. Chemical Modification (Functionalization) of the Internal Surface of Apoferritin Nanoparticles

Apoferritin nanoparticles in accordance with one embodiment of the invention were produced by chemical modification of the internal surface with small hydrophobic molecules. In this way, the protein was genetically modified in a manner such that the amino acid cysteine was only present at specific exposed sites on the internal surface. This amino acid is the only amino acid in the internal void to have a thiol group in its side chain, which was used as an anchor site for the reaction with hydrophobic molecules. The protein container can be fragmented into its subunits under acidic conditions (pH of 2). The internal cysteines can then be functionalized with the hydrophobic molecules. The molecules 2-iodo-N-phenylacetamide and 2-bromo-N-decylacetamide were used, which can bind to the thiol group following a substitution reaction. Because a thiol group only occurs in cysteine and the modified protein contains this only inside the cavity, then only the internal surface is selectively modified with hydrophobic groups. The complete functionalization can be verified using ESI-MS measurements by a significant increase in the mass of the protein. This can also be used to determine whether every cysteine has been functionalized. Heretofore, up to 96 hydrophobic molecules per nanoparticle could be incorporated. After the functionalization, an enhanced adsorption capacity for the IS and pCS as well as for phenyl acetate could be measured, as can be seen in FIG. 3a.

### Materials and Methods

### General

All of the chemicals were obtained from commercial sources and were used without further purification. As far as possible, all of the solutions were produced with ultrapure water (produced with a Purelab-Flex-2-System, specific resistance 18.2 M (2 cm) and reagents in analytical quality, unless stated otherwise.

### Mutagenesis

The introduction of cysteine anchor sites was carried out by several cycles of site-directed QuikChange™ mutagenesis using a two-stage polymerase chain reaction (PCR) protocol [30]. The primers used for the various mutation sites are given in Table 1.

TABLE 1
Primer sequences (in 5′-3′-direction) for PCR
mutagenesis protocol

| | | SEQ ID |
|---|---|---|
| Mutation | Forward primer | NO: |
| C130A | CAAGAACGATCCGCATCTGGCCGATTTCATC | 3 |
| | GAAACCCAC | |
| K53C | TGTTGCACTGAAGAACTTTGCGTGTTACTTT | 4 |
| | CTGCATCAGTCCCATG | |
| E64C | TTTCATCAGTTTCTCGGCATGACAGCGTTCT | 5 |
| | TCATGGGACTGATG | |
| K143C | ATCCCCGAGTTCTTTGATCGCACAGACCTGT | 6 |
| | TCGTTCAGATAGT | |
| S178C | CACACTTTGGGTGATTGCGATAATGAATCGT | 7 |
| | AACTCGAGCACC | |

A mixture of 2.9 μL of pET-22b (+) plasmid containing the gene of interest (7 ng μL⁻¹), 1 μL 10 of mM dNTP-Mix, 5 μL of reaction buffer 10× (100 mM KCl, 100 mM (NH₄)₂SO₄, 200 mM Tris-HCl pH 8.8, 20 mM MgSO₄, 1% Triton® X-100, 1 mg mL⁻¹ of nuclease-free bovine serum albumin (BSA)), 1 μL of Pfu-DNA polymerase (2.5 U μL⁻¹) and 38.1 μL of ultrapure water was produced. The mixture was halved and 1 μL of forward primer or reverse primer (10 μmol μL⁻¹) was added to each tube. A PCR thermocycler (Eppendorf Mastercycler Nexus PCR Cycler) was prepared with an initial heating step for 30 s at 95 °C. The first step of the PCR protocol consisted of 3×30 s cycles of denaturing at 95 °C, a constant temperature stage for 1 min at 61 °C, followed by elongation for 6 min at 68 °C. After the first 3 cycles, the separate mixtures were combined with the forward primer and reverse primers and the PCR was continued for 16 further cycles with the same parameters as in the first 3 cycles, followed by a final elongation phase for 10 min at 68 °C to complete the PCR. Digestion of the parental plasmid was carried out by adding 1 μL of DpnI (10 U μL⁻¹) and incubating overnight at 37 °C DpnI was deactivated by heating for 20 min at 80 °C and the mixture was purified with the NucleoSpin® Gel and PCR Clean-Up-Kit, following the instructions of the manufacturer. Calcium-competent E. coli DH5α cells were incubated for 30 min on ice with 200 ng of the purified plasmid, followed by a thermal shock for 45 s at 42 °C Next, the cells were incubated for 1 h in “Super Optimal Broth” (SOB) medium, centrifuged at 1000 g, resuspended in 100 μL of medium, plated out onto a LB agar plate and incubated for 16 h at 37 °C. An individual colony was picked up and incubated overnight at 37 °C and at 250 rpm in 5 mL of sterile LB medium, supplemented with 150 μg mL⁻¹ of ampicillin. The next day, the plasmids were extracted with the NucleoSpin® plasmid Miniprep-Kit, following the instructions of the manufacturer.

The sequence was confirmed by mixing 500 ng of plasmid with 25 μmol of T7 forward primer or reverse primer in 10 μL of solution and sending it for DNA sequencing (Eurofins Genomics). Plasmids with the desired mutations were then selected as stock plasmids for further mutagenesis, until all 5 mutations were present.

### Production and Purification of Ferritin-Cysteine Variants

The production of negatively charged ferritin variants (Ftn (neg)) and variants carrying cysteine was carried out as already published [31]. Firstly, calcium-competent E. coli BL21-Gold (DE3) cells were thawed for 10 min on ice. Next, 1 μL of 40 ng μL⁻¹ plasmid solution was added to the cells and the mixture was incubated on ice for 30 min. Thermal shock was carried out by incubating the mixture at 42 °C for 45 s, followed by 2 min of incubation on ice. The cells were suspended in 1 mL of SOB medium and incubated for 1 h at 37 °C The cells were centrifuged at 1000 g and 1 mL of the mixture was removed. The cell pellet was resuspended in the remaining solution and spread onto a LB agar plate with 150 μg mL⁻¹ of ampicillin and incubated overnight at 37 °C. In order to produce the precultures, colonies of transformed E. coli BL21-Gold (DE3) cells (Agilent) were incubated overnight in 5 mL of sterile LB-Miller medium, supplemented with 150 of μg mL⁻¹ sodium-ampicillin, at 37 °C and 180 rpm. Next, 400 mL of Terrific Broth (TB) medium with 150 μg mL⁻¹ of sodium-ampicillin was inoculated with 4 mL of the preculture. The cells were grown at 37 °C and 180 rpm, until an OD₆₀₀ of 0.6 was reached. Protein over-expression was induced by adding isopropyl-β-D-1-thiogalactopyranoside (IPTG) in a final concentration of 0.25 mM, and the cells were incubated for a further 48 h at 18 °C. The cells were harvested by centrifuging at 4000 g. Pellets were stored at −20 °C until further use.

Cells from 400 mL of culture were resuspended in 20 mL of buffer (50 mM Tris, pH 7.5, 0.3 M NaCl). Cell lysis was obtained by sonication (60% amplitude) for six times 1 min on ice with a 1 min pause in between, using a Vibra-Cell VCX-130 ultrasonic processor (Sonics). The resulting suspension was centrifuged at 14000 g for 20 min, in order to separate the cell debris from the soluble proteins. Denaturing of most of the E. coli proteins was obtained by heating the supernatant to 65 °C for 10 min in a water bath. The denatured proteins were separated by centrifuging for 15 minutes at 14000 g. Proteins remaining in the solution were precipitated out with ammonium sulfate at a final concentration of 70% of its saturation concentration, followed by centrifuging at 14000 g for 20 minutes. After rebuffering the pellet in 10 mL of buffer (50 mM Tris, pH 7.5, 0.15 M NaCl), the ammonium sulfate precipitation was repeated. The resulting pellet was dissolved in 50 mL of IEC loading buffer (50 mM Tris, pH 7.5, 0.15 M NaCl) and purified by ion exchange chromatography (IEC) with a linear gradient of 0.15 to 1 M NaCl using a 5 mL HiTrap™ Q HP anion exchange column (Cytiva). All of the Ftn^{(neg)}-4xCys-containing fractions were collected and concentrated with a Sartorius Vivaspin® Turbo 15 (MWCO 30000) filtration unit to an end volume of 2 mL. Finally, the sample was purified by gel filtration with a HiLoad 16/600 Superdex™ 200 μg column. All of the chromatography steps were carried out on an Akta pure system from Cytiva. All of the fractions containing Ftn^{(neg)}-4xCys were collected and stored at 4 °C until further use.

Functionalization with 2-Iodo-N-Phenylacetamide

5 mg of Ftn^{(neg)}-3xCys or Ftn^{(neg)}-4xCys was incubated for 4 h in disassembly buffer (10 mM phosphate; 50 mM NaCl, pH 2). After 3½ h, 10 equivalents (eq.) (with respect to each cysteine) of tris(2-carboxyethyl)phosphine hydrochloride (TCEP, Iris Biotech GmbH) from a 10 mg mL⁻¹ stock solution was added to the solution. Next, the solution was made up with reassembly buffer (50 M Tris, 50 mM NaCl, pH 7.6) to 15 mL and concentrated with a membrane filter (Sartorius Vivaspin Turbo 15; 30 kDa MWCO) to an end volume of 200 μL. Another 10 eq. of TCEP was added and the solution was made up to a volume of 2 mL. The pH was adjusted to 7.6 with 1M NaOH or HCl. Next, 2 mL of ethanol with 20 eq. of 2-iodo-N-phenylacetamide (abcr GmbH) was added to the solution and the mixture was stirred for 1 h at 300 rpm in the dark. Next, the solution was made up to a total volume of 30 mL with reassembly buffer. The protein reassembled itself overnight. Finally, the solution was concentrated to 2 mL and purified by gel filtration on a HiLoad 16/600 Superdex™ 200 μg column. Protein-containing fractions were collected and stored at 4 °C for further use.

Functionalization with 2-Bromo-N-Decylacetamide

The functionalization followed exactly the same protocol as for the functionalization with 2-iodo-N-phenylacetamide. However, during the functionalization reaction, the protein/TCEP solution was not made up to 2 mL, but to 800 μL, and then 3.2 mL of ethanol, which contained 40 eq. of 2-bromo-N-decylacetamide (Sigma-Aldrich), was added to the solution. All of the other steps were carried out in accordance with the protocol for 2-iodo-N-phenylacetamide.

### Hanging Drop Crystallization

The crystallization of small quantities of protein or functionalized protein variants was carried out using the hanging drop vapor diffusion technique. Reservoir solution (100 mM Tris, 500 mM MgOAc, pH 8.5) was produced in a manual 24-well plate. Drops were produced on siliconized cover slides (Jena Bioscience) by mixing 2 μL of reservoir solutions with 1 μL of 50 mM Tris, 1 M NaCl, pH 7.5 buffer and 1 μL of the respective ferritin variant. The plates were incubated at 25 °C. The first crystals were visible after one day.

### Batch Crystallization

In order to crystallize larger quantities of Ftn^{(neg)} and functionalized variants of Ftn (neg), batch crystallization strategies based on a protocol by Rayment were used [32]. For a standard experiment, 250 μL of a 50 mM Tris 1 M NaCl pH 7.5 buffer was carefully mixed with identical quantities of Ftn^{(neg)} stock solution with a concentration of 12 mg mL⁻¹ in 50 mM Tris 0.3 M NaCl pH 7.5 buffer. Next, 500 μL of the precipitation solution (133 mM Tris, 333 mM MgOAc, pH 8.5) was added dropwise, with constant shaking. The mixture was stored for 7 days without stirring at an ambient temperature of 20 °C before the crystals were fixed.

### Crystal Fixing

An increase in the stability of the crystals was required for the adsorption experiments. To this end, they were fixed with the crosslinker Sulfo-SMCC (sulfosuccinimidyl 4-(N-maleimidomethyl)cyclohexane-1-carboxylate, Sigma-Aldrich). The crystals were centrifuged off for 2 min at 1000 g. In a standard test with a total mass of 3 mg of crystals, the crystallization solution was removed until 246 μL remained. Next, 64 μL of a freshly prepared 4.8 mg mL⁻¹ aqueous Sulfo-SMCC solution was added, resulting in a final concentration of 1 mg mL⁻¹. The mixture was left to stand for 4 h at ambient temperature and then made up to 1 mL with ultrapure water. The crystals were centrifuged off by centrifuging at 1500 g for 2 min. The supernatant was removed and the crystals were then resuspended in ultrapure water. The method was repeated up to three times in order to wash the crystals free from the remaining crosslinking agent. The material was stored until further use at an ambient temperature of 20 °C Crystals were photographed with a FlexaCamC1 under a Leica S9D microscope.

For fixing with glutaraldehyde (Merck), 50 μL of a 2.5% aqueous glutaraldehyde solution was added to 1 mL of crystal solution which contained 3 mg of crystals, resulting in an end concentration of 0.00119%. The crystals were incubated for 4 h and then washed three times with ultrapure water. The crystals were stored at 20 °C until further use.

After the glutaraldehyde crosslinking, the crystals still dissolved in a 60 mg mL⁻¹ BSA solution. The stability could be enhanced by an additional fixing step. The procedure was repeated, but prior to washing, the crystals were incubated for only 10 min. Toxin adsorption assays showed a significantly reduced adsorption capacity, however. It was assumed that the polymerization of the glutaraldehyde led to blocking of the pores.

### Production of Non-Crystalline Adsorbent

500 μL of a 6 mg mL⁻¹ protein solution was supplemented with 25 μL of 2.5% glutaraldehyde solution. Complete mixing was ensured by careful forward pipetting and reverse pipetting of the solution three times. The mixture was kept overnight at an ambient temperature of 20 °C. The next day, a white precipitate had formed. The material was washed three times with ultrapure water and stored at 20 °C until further use.

### ESI-MS Measurements

The protein sample was rebuffered with an Amicon® Ultra 0.5 mL (MWCO 30000 Da) centrifugal filter using ultrapure water. The protein sample was made up to a volume of 500 μL with ultrapure water, concentrated to approximately 20 μL and then made up again to 500 μL. The rebuffering was repeated 5 times and the concentration was adjusted to between 0.15 and 0.2 mg mL⁻¹. The mass of the proteins was determined with electrospray ionization-time of flight mass spectrometry (Agilent 6224 ESI-TOF). The measurement was carried out in positive mode.

### Toxin Assay

Adsorption experiments were carried out in order to determine the uremic toxin adsorption capacity of the ferritin variants. All of the solutions and samples were handled in glassware (Macherey-NAGEL Vials N9), because in initial experiments, an adsorption of the toxins onto the polypropylene walls of the reaction tubes was observed.

Firstly, a stock solution of the desired toxin was produced with a concentration of 50 μg mL⁻¹ for pCS and IS and 500 μg mL⁻¹ for PheAc. A calibration series with concentrations of 0.01, 0.05, 0.1, 0.2, 0.5, 0.7 and 1 μg mL⁻¹ was produced for later determination of the absolute toxin concentrations in the samples.

The stock solutions were diluted further in order to obtain the final uremic toxin concentrations which would be expected in a stage 5 CKD patient (41 mg L⁻¹ for pCS, 44 mg L⁻¹ for IS and 474 mg L⁻¹ for PheAc [34]). The adsorbent was centrifuged at 1500 g for 2 min and the entire supernatant was removed from the sample. 150 μL of the respective toxin solution was added and the crystals were incubated for 3 h at ambient temperature. In addition, 150 μL of the toxin solution was incubated as the control. Three 10 μL aliquots were removed from all of the samples and diluted 100-fold in ultrapure water. Finally, the crystals were washed with water, vacuum dried and weighed.

The uremic toxin concentration was quantified by means of a reverse phase high performance liquid chromatography system (RP-HPLC) with a C18 column (Zorbax Extend-C18, Agilent), coupled to an electrospray ionization quadrupole linear ion trap mass spectrometer (ESI-QTRAP). The solvent used was a mixture of HPLC quality water (LiChrosolv®, Merck) and acetonitrile (LiChrosolv®, Merck), both with the addition of 0.1% formic acid (Honeywell Fluka). The specific compositions in each step during the 15-minute chromatography program are summarized in Table 2.

TABLE 2
Sequence of HPLC program

| Time | H₂O | Acetonitrile | Flow rate |
|---|---|---|---|
| [min] | [%] | [%] | [mL min⁻¹] |
| 0.00 | 97 | 3 | 0.3 |
| 8.00 | 70 | 30 | 0.3 |
| 9.00 | 40 | 60 | 0.3 |
| 10.50 | 97 | 3 | 0.3 |

The control was measured before each sample incubated with the crystals. Chromatograms were analyzed with the Analyst® Instrument Control and Data Processing software. Peaks were integrated and the toxin concentration was determined from the calibration series. The quantity of adsorbed uremic toxin was determined from the difference in concentration between the control and the samples. The adsorption capacity was then determined by dividing the mass of the adsorbed toxins by the mass of the crystals.

Quantitative polymerase chain reaction analysis (qPCR) of mRNA expression in endothelial cells of the human aorta

Human aorta endothelial cells (hAoECs) (Promocell) were cultured in an Endothelial Cell Growth Medium MV (Promocell). The cells were seeded into 24-well plates (15×10⁴ cells/well) at 80% confluence and incubated for 6 h with 100 ng mL⁻¹ of lipopolysaccharides (LPS) or functionalized or non-functionalized protein crystals. After the incubation period, the total RNA was extracted using the RNAeasy-Minikit (Qiagen). The reverse transcription was carried out using 1 μg of total RNA (600 ng), random hexamer primers and Verso-Reverse-Transcriptase (Thermo Scientific) following the instructions of the manufacturer. For the real-time PCR, the gene expression level was quantified using SYBR Green I dye on a LightCycler 480 system (Roche Applied Sciences). The following primers were used for the relative quantification of the targeted gene expression—for human TNF-alpha: Forward primer 5′-GCCCAGGCAGTCAGATCATCT-3′ (SEQ ID NO: 8), Reverse primer 5′-TTGAGGGTTTGCTACAACATGG-3′ (SEQ ID NO: 9) and for human beta actin: Forward primer 5′-CAACCGCGAGAAGATGAC-3′ (SEQ ID NO: 10), Reverse primer 5′-GTCCATCACGATGCCAGT-3′ (SEQ ID NO: 11). The data were given as the mean degree of gene expression relative to the expression of the reference gene (β-actin).

### Blood Platelet Activation Assay

The blood platelets from three donors were isolated by centrifuging for 15 minutes at 260 g. After a second centrifugation step, the blood platelets were resuspended in Hepes buffer, pH 6.6 (10 mM Hepes, 136 mM NaCl, 2.7 mM KCl, 2 mM MgCl₂ and 5 mM glucose). Thrombocyte suspensions were centrifuged again in the presence of 1:15 acid citrate dextrose (ACD) and 1 U mL⁻¹ of apyrase and then resuspended in Hepes buffer pH 7.45 (10 mM Hepes, 136 mM NaCl, 2.7 mM KCl, 2 mM MgCl₂, 5 mM glucose and 0.1% BSA). 15×10⁶ blood platelets were incubated for 15 min with 4 nmol L⁻¹ of thrombin in the presence of 2 mmol L⁻¹ CaCl₂) or various protein crystals. The thrombocytes were lysed with 4% SDS lysis buffer (200 mmol L⁻¹ Tris, 600 mmol L⁻¹ NaCl, 4% SDS) including EDTA-free Halt Protease Inhibitor Cocktail (1:10; Sigma-Aldrich) and Halt Phosphatase Inhibitor Cocktail (1:10; Sigma-Aldrich). The protein quantity was quantified in accordance with the protocol for the DC protein assay (Bio-Rad). An identical quantity of protein from each sample was separated by means of 10% SDS-polyacrylamide gel electrophoresis, transferred onto nitrocellulose membranes and blocked with 5% bovine serum albumin (BSA) for 1 h at ambient temperature. Anti-p-Akt antibody (1:1000; cell signaling) and anti-tubulin antibody (1:1000; cell signaling) were used as the primary antibodies. The blots were incubated overnight at 4 °C A second anti-rabbit antibody (1:1000; cell signaling) was used and incubated for 1 h at ambient temperature. Immunoreactive bands were made visible by reinforced chemiluminescence and densitometry was carried out using “Quantity One Software” (Bio-Rad Laboratories).

### Sequences

The amino acid sequences of the Ftn^{(neg)} nanoparticle subunits and the cysteine-containing variants Ftn^{(neg)}-3xCys (with 3 cysteine residues) and Ftn^{(neg)} 4xCys (with 4 cysteine residues) are given below. Variants with only one (Ftn^{(neg)}-1xCys) as well as two cysteine residues (Ftn^{(neg)}-2xCys) are also given. For Ftn (neg), the mutations given are with respect to the wild-type H-chain (SEQ ID NO: 1). Mutations compared with Ftn^{(neg)} (SEQ ID NO: 12), including originally present cysteine residues, which were modified to alanine residues, are given. The cysteine residues in the cysteine-containing variants are highlighted by underlining.

Ftn^{(neg)}, (SEQ ID NO: 12): Variant with increased
negative charge on the external surface;
A18E, C90E, C102E, K86Q, H105E
TTASTSQVRQNYHQDSEEAINRQINLELYASYVYLSMSYYFDRDDVALKN
FAKYFLHQSHEEREHAEKLMKLQNQRGGRIFLQDIQKPDEDDWESGLNAM
EEALELEKNVNQSLLELHKLATDKNDPHLCDFIETHYLNEQVKAIKELGD
HVTNLRKMGAPESGLAEYLFDKHTLGDSDNES
Ftn^{(neg)}-3xCys (SEQ ID NO: 13): Variant with 3
cysteine residues on the internal surface;
K53C, E64C, C130A, K143C
TTASTSQVRQNYHQDSEEAINRQINLELYASYVYLSMSYYFDRDDVALKN
FACYFLHQSHEERCHAEKLMKLQNQRGGRIFLQDIQKPDEDDWESGLNAM
EEALELEKNVNQSLLELHKLATDKNDPHLADFIETHYLNEQVCAIKELGD
HVTNLRKMGAPESGLAEYLFDKHTLGDSDNES
Ftn^{(neg)}-4xCys (SEQ ID NO: 14): Variant with 4
cysteine residues on the internal surface; K53C,
E64C, C130A, K143C, S178C
TTASTSQVRQNYHQDSEEAINRQINLELYASYVYLSMSYYFDRDDVALKN
FACYFLHQSHEERCHAEKLMKLQNQRGGRIFLQDIQKPDEDDWESGLNAM
EEALELEKNVNQSLLELHKLATDKNDPHLADFIETHYLNEQVCAIKELGD
HVTNLRKMGAPESGLAEYLFDKHTLGDCDNES
Ftn^{(neg)}-1xCys (SEQ ID NO: 15): Variant with 1
cysteine residue on the internal surface;
K53C, C130A
TTASTSQVRQNYHQDSEEAINRQINLELYASYVYLSMSYYFDRDDVALKN
FACYFLHQSHEEREHAEKLMKLQNQRGGRIFLQDIQKPDEDDWESGLNAM
EEALELEKNVNQSLLELHKLATDKNDPHLADFIETHYLNEQVKAIKELGD
HVTNLRKMGAPESGLAEYLFDKHTLGDSDNES
Ftn^{(neg)}-2xCys (SEQ ID NO: 16): Variant with 2
cysteine residues on the internal surface;
K53C, E64C, C130A
TTASTSQVRQNYHQDSEEAINRQINLELYASYVYLSMSYYFDRDDVALKN
FACYFLHQSHEERCHAEKLMKLQNQRGGRIFLQDIQKPDEDDWESGLNAM
EEALELEKNVNQSLLELHKLATDKNDPHLADFIETHYLNEQVKAIKELGD
HVTNLRKMGAPESGLAEYLFDKHTLGDSDNES

Sequences of other variants which were investigated are given below. Mutations compared with Ftn^{(neg)} (SEQ ID NO: 12) are given. The term “binding site” stands here for toxin binding sites, which have been formed by simple amino acid exchange (without additional functionalization).

Ftn^{(neg)}-Ap4 (SEQ ID NO: 17): Variant with reduced
negative surface charge in the region of the
internal void; E61V, E62A, D131F, E140W
TTASTSQVRQNYHQDSEEAINRQINLELYASYVYLSMSYYFDRDDVALKN
FAKYFLHQSHVAREHAEKLMKLQNQRGGRIFLQDIQKPDEDDWESGLNAM
EEALELEKNVNQSLLELHKLATDKNDPHLCFFIETHYLNWQVKAIKELGD
HVTNLRKMGAPESGLAEYLFDKHTLGDSDNES
Ftn^{(neg)}-Ap4-3A (SEQ ID NO: 18): Variant with
reduced negative surface charge in the region of
the internal void and at the pores, as well as
enlarged pore size; E61V, E62A, T122A, D123A,
N125A D131F, E140W
TTASTSQVRQNYHQDSEEAINRQINLELYASYVYLSMSYYFDRDDVALKN
FAKYFLHQSHVAREHAEKLMKLQNQRGGRIFLQDIQKPDEDDWESGLNAM
EEALELEKNVNQSLLELHKLAAAKADPHLCFFIETHYLNWQVKAIKELGD
HVTNLRKMGAPESGLAEYLFDKHTLGDSDNES
Ftn^{(neg)}-Ap7 (SEQ ID NO: 19): Variant with 7
hydrophobic amino acids on the internal surface;
E61V, E62A, H128F, D131W, N139V, E140W, K143V
TTASTSQVRQNYHQDSEEAINRQINLELYASYVYLSMSYYFDRDDVALKN
FAKYFLHQSHVAREHAEKLMKLQNQRGGRIFLQDIQKPDEDDWESGLNAM
EEALELEKNVNQSLLELHKLATDKNDPFLCWFIETHYLVWQVVAIKELGD
HVTNLRKMGAPESGLAEYLFDKHTLGDSDNES
Ftn(^{neg)}-Ap16 (SEQ ID NO: 20): Variant with 16
hydrophobic amino acids on the internal surface;
K49V, Y54F, H57W, Q58L, E61V, E62A, H65L, H128F,
H136Y, D131W, H136Y N139V, E140W, K143V, E147L,
H151Y, N154A
TTASTSQVRQNYHQDSEEAINRQINLELYASYVYLSMSYYFDRDDVALVN
FAKFFLWLSHVARELAEKLMKLQNQRGGRIFLQDIQKPDEDDWESGLNAM
EEALELEKNVNQSLLELHKLATDKNDPFLCWFIETYYLVWQVVAIKLLGD
YVTALRKMGAPESGLAEYLFDKHTLGDSDNES
Ftn^{(neg)}-dock03 (SEQ ID NO: 21): Variant with a
binding site at the internal surface in the
vicinity of the threefold channel; E64R, H65K,
K68E, K71R, Q75D, R76K, H128E, D131E, F132H,
T135K, H136R, Y137H, N139R, E140R
TTASTSQVRQNYHQDSEEAINRQINLELYASYVYLSMSYYFDRDDVALKN
FAKYFLHQSHEERRKAEELMRLQNDKGGRIFLQDIQKPDEDDWESGLNAM
EEALELEKNVNQSLLELHKLATDKNDPELCEHIEKRHLRRQVKAIKELGD
HVTNLRKMGAPESGLAEYLFDKHTLGDSDNES
Ftn^{(neg)}-dock23 (SEQ ID NO: 22): Variant with a
binding site at the internal surface in the center
of the subunit; H57E, Q58R, H60I, E61G, E64G,
K68D, T135D, H136K, Y137H, N139E, E140K, A144N
TTASTSQVRQNYHQDSEEAINRQINLELYASYVYLSMSYYFDRDDVALKN
FAKYFLERSIGERGHAEDLMKLQNQRGGRIFLQDIQKPDEDDWESGLNAM
EEALELEKNVNQSLLELHKLATDKNDPHLCDFIEDKHLEKQVKNIKELGD
HVTNLRKMGAPESGLAEYLFDKHTLGDSDNES
Ftn^{(neg)}-dock43 (SEQ ID NO: 23): Variant with a
binding site at the internal surface in the
vicinity of the E helix; H57E, Q58R, E64R, H65K,
K68E, T135D, H136R, Y137H, N139R, E140R, E147Y
TTASTSQVRQNYHQDSEEAINRQINLELYASYVYLSMSYYFDRDDVALKN
FAKYFLERSHEERRKAEELMKLQNQRGGRIFLQDIQKPDEDDWESGLNAM
EEALELEKNVNQSLLELHKLATDKNDPHLCDFIEDRHLRRQVKAIKYLGD
HVTNLRKMGAPESGLAEYLFDKHTLGDSDNES
Ftn^{(neg)}-Ap4-3A-dock43 (SEQ ID NO: 24): Variant
with reduced negative surface charge in the
region of the internal void and at the pores, as
well as enlarged pore size, and in addition with
a binding site at the internal surface in the
vicinity of the E helix; E61V, E62A, T122A,
D123A, N125A, D131F, H57E, Q58R, E64R, H65K,
K68E, T135D, H136R, Y137H, N139R, E140R, E147Y
TTASTSQVRQNYHQDSEEAINRQINLELYASYVYLSMSYYFDRDDVALKN
FAKYFLERSHVARRKAEELMKLQNQRGGRIFLQDIQKPDEDDWESGLNAM
EEALELEKNVNQSLLELHKLAAAKADPHLCFFIEDRHLRRQVKAIKYLGD
HVTNLRKMGAPESGLAEYLFDKHTLGDSDNES

### Results:

Assembly into a macroscopic material with the apoferritin nanoparticle as the building block was achieved by batch crystallization techniques (see above). A protein solution was carefully mixed with constant shaking while the precipitation agent solution was being added. The first crystals could be seen after approximately 24 h. The size of the crystals could be adjusted by varying the protein and precipitation agent concentration. In order to increase the stability, the crystals were fixed with a crosslinker. Initial experiments were carried out with glutaraldehyde. In stability tests in a 60 mg mL⁻¹ BSA solution, which was selected in order to simulate the high protein content of blood, however, the crystals dissolved. Thus, stabilization by means of a Sulfo-SMCC crosslinker was carried out, enabling stable fixing by maintaining the adsorption capacity.

For better adsorption of hydrophobic and in part negatively charged PBUTs, hydrophobic ligands were introduced into the internal void of the apoferritin nanoparticles in accordance with the invention. The invention provides a modular material which can be adapted to the nature of the ligands. To this end, the incorporation of cysteine residues, which can be modified because their thiol group serves as an anchor site for chemical derivatization, provides a generic site for modification. On the other hand, native cysteine residues were replaced with alanine residues in order to avoid modification at unwanted positions. Important design criteria for the introduction of cysteine residues on the cavity surface were the distance between the sites and the solvent-accessible surface area (SASA). A high SASA value should correspond to a high reactivity. To this end, ferritin variants with three and four introduced cysteines per subunit with the designations Ftn^{(neg)}-3xCys and Ftn^{(neg)}-4xCys were designed. The total number of anchor sites per assembled protein cage in the case of 24 subunits and 3 or 4 cysteine residues was therefore 72 or 96. The mutations were introduced into the Ftn^{(neg)} gene with Quik-Change mutagenesis techniques and the variants were over-expressed in E. Coli bacteria. Purification followed the published protocol for Ftn^{(neg)} [31]. In ion exchange chromatography (IEC) and size exclusion chromatography (SEC), compared with the starting protein, no notable variation in the elution behavior was observed. The introduction of the desired mutations was verified by electrospray ionization mass spectrometry (ESI-MS), wherein the found mass corresponded to the calculated mass (not shown).

For the chemical modification of cysteine thiol groups, the α-halogenocarbonyls 2-iodo-N-phenylacetamide (Phe)

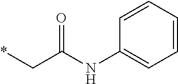 [SMILES: *CC(=O)Nc1ccccc1]

and 2-bromo-N-decylacetamide (C10)

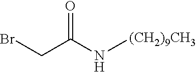

were used. Apoferritin nanoparticles that have been functionalized with the above compounds are designated as Ftn^{(neg)}-Phe and Ftn^{(neg)}-C10. The overall strategy for the chemical modification of the internal surface and the subsequent assembly to form the heterogenic material is in principle as follows: firstly, the protein cage is disassembled into its subunits under acid conditions. In the disassembled state, the thiol groups are readily accessible to the halogenoacetamide derivatives. The molecules to be bound per se are not soluble in an aqueous solution. Thus, the functionalization is carried out in a solution which contains high proportions of ethanol in order to facilitate the solubility of the hydrophobic molecules. After incubation of the reaction partner, the mixture is diluted and the protein cage can then be reassembled. After purification by SEC, the resulting chromatogram shows a similar elution volume to that of the non-functionalized cage (not shown), which indicates that the protein cage has been fully reconstructed. This finding is confirmed in negatively stained TEM images, which indicates intact cage structures (not shown). The SEC of functionalized Ftn^{(neg)}-4xCys exhibits a slight shift to a higher elution volume, linked to a slightly increased size, which agrees well with the results of the dynamic light scattering measurements (DLS).

In order to check the successful and complete functionalization of the cysteine anchor sites, ESI-MS measurements were carried out. In initial tests, several mass peaks per charged species could be observed (not shown). Each of them could be allocated to the ferritin subunit with one to four of the desired molecules, which indicates that a mixture of different degrees of functionalization was present in the sample. The fine-tuning of the reaction conditions, in particular the proportion of ethanol in the mixture, the ratio of reactive molecules to cysteines and the addition of a reducing agent (TCEP), allowed for a reproducible, complete functionalization of all sites, as could be confirmed by ESI-MS (not shown). The derivatization was also demonstrated using X-ray crystallography of the functionalized ferritin (not shown). Finally, macroscopic crystalline materials from the functionalized variants were produced under the same conditions as described above, which indicates that the functionalization had not affected the external surface.

Next, PBUT adsorption assays were carried out with the non-functionalized protein-based material and the material functionalized with aliphatic or phenyl molecules. To this end, the respective sample was incubated in solutions of three different uremic toxins, indoxyl sulfate (IS), p-cresyl sulfate (pCS) and phenylacetic acid (PheAc) in concentrations which are expected in end-stage CKD patients [26, 33, 34]. After incubation for 3 h, the PBUT concentration in the supernatant and the respective control samples was determined using HPLC-MS/MS techniques. The determination of the absolute values was carried out by comparison with a calibration experiment, which was carried out before the start of every experimental run and every 20 samples. In addition, controls were measured shortly before every sample, in order to ensure a direct comparability. One challenge was the non-specific adsorption of IS onto the flasks of reaction vessels produced from polymers which, however, could be overcome by switching to glass flasks for the incubation and storage of the samples. Finally, the protein-based material was vacuum dried and weighed in order to determine the mass. The adsorption capacity, i.e., the ratio of the adsorbed PBUT mass to the total mass of the material, was calculated and is shown in FIGS. 4a-c.

Crystals of the non-functionalized protein Ftn^{(neg)} adsorbed all three tested toxins with a capacity of between 247 and 283 μg g⁻¹ for pCS and IS and 2710 μg g⁻¹ for PheAc. The reason for the significantly higher capacity for the PheAc adsorption is most likely the 10-fold higher concentration of the PBUTs in the assay. In the case of IS and pCS, the functionalization with phenyl molecules (Phe) led to an increase in the adsorption capacity to 458 or 372 μg g⁻¹. For the functionalization with the aliphatic molecules (C10), increased adsorption could only be observed for IS (FIG. 4a). For the PheAc toxin, after incorporation of the hydrophobic molecules, no significant increase in the adsorption could be observed. In order to test whether a highly-ordered material with a uniform distribution of solvent channels and pores was advantageous to adsorption, the material was compared with samples of non-crystalline protein material which was produced by adding a crosslinker to a protein solution and incubation overnight (not shown). The resulting material was tested in respect of its adsorption capacity for IS. No noticeable difference between the two types of material could be found, which indicates that the macroscopic form of the material does not influence the adsorption of the IS. The capacities of the protein-based adsorbent in accordance with the invention lie in a similar range, but are smaller than the values for other published materials, for example P87 zeolite with capacities of up 1000 μg mL⁻¹ or carbon-based adsorbents with capacities of up to 3200 μg mL⁻¹ in respect of IS. To the knowledge of the inventors, the adsorbent with the highest published capacity thus far is a zirconium-based MOF with a capacity of up to 156 mg g⁻¹ [24]. However, these materials are good adsorbents per se, and efforts have to be made to improve their biocompatibility. In comparison, the protein-based material in accordance with the invention has an intrinsic biocompatibility. The adsorption capacity can be adjusted and further improved.

In order to demonstrate the biocompatibility of the material in accordance with the invention, endothelial cells and isolated blood platelets were incubated with the crystalline material. Endothelial cells exhibited no expression of tumor necrosis factor α (TNF-α), indicative of no endotoxin contamination by the bacterial origin of the material (FIG. 4d). Blood platelets were not activated, which indicates that the material does not induce blood coagulation (FIG. 4c). Similar results were obtained for crystals crosslinked with glutaraldehyde (not shown). In addition, no great difference was observed between the chemically modified and unmodified protein materials, which indicates that the functionalization had not affected the biocompatibility.

In total, the material synthesized in accordance with the invention, based on bottom-up assembly of protein cages, has been shown to be highly suitable for blood purification applications. The resulting material exhibits stability in blood-like systems and good adsorption of three PBUTs. Crystalline and non-crystalline adsorbents behave in a similar manner. Furthermore, it has been shown that by introducing anchor sites, up to 96 water-insoluble aliphatic and phenyl molecules can be incorporated into the void of the ferritin protein cage. After modification, no reduction in biocompatibility could be observed. An increase in the adsorption capacity due to the chemical modifications should be observed. The adsorption capacity could very probably be increased still further by providing ligands other than exclusively hydrophobic ligands, for example amphiphilic ligands, a mixture of hydrophobic and hydrophilic ligands or the modification to the amino acid residues in the environment of the ligands, because PBUTs have hydrophobic and hydrophilic properties. The modular character of the material in accordance with the invention allows for an adaptation to other applications, for example treatment strategies in heavy metal contamination by incorporating chelating agents. Furthermore, by means of genetic modifications, positively charged amino acids can be introduced around the anchor sites in order to compensate for the negative charge of the PBUTs, while the hydrophobic portion of the toxins is bonded to the molecules that have been introduced. All of these modifications to the internal surface have no influence on the assembly of the material.

2. Modification of the Internal Surface and of the Channel Region of Apoferritin Nanoparticles in Accordance with the Invention (without Additional Chemical Functionalization).

An efficient adsorption to the apoferritin nanoparticles in accordance with the invention can also be obtained by a suitable combination of amino acids with hydrophobic, aromatic, polar or charged side chains, whereupon binding sites inside the protein container can even be created in non-functionalized proteins. This is shown by way of example in FIG. 5b. Here, the delocalized and hydrophobic ring system of the IS is bound to the ring system of the amino acid tyrosine, while the polar negatively-charged sulfate group is stabilized by partially positively-charged nitrogen atoms in the amino acid histidine. In order to design these protein variants, the binding affinity of the proteins to the toxins was determined with the aid of “ligand docking” protocols from the Rosetta software package. Next, various amino acids in the protein were randomly varied in the computer model and the affinity was determined once again. This procedure was repeated until no further increase in the affinity was observed.

The protein containers designed in this manner were produced in the laboratory, they were still soluble and they assembled into complete containers. The mutations which were introduced were confirmed by ESI-MS measurements. Variants with increased affinity for IS were designed and successfully produced. Adsorbents from these variants to some extent exhibited a significantly increased adsorption capacity as regards the appropriate toxin, as can be seen in FIG. 5a (Ftn^{(neg)}-Dock=Ftn^{(neg)}-dock03, SEQ ID NO: 21).

Furthermore, amino acids of the apoferritin nanoparticle can be specifically exchanged in order to vary the properties of the apoferritin nanoparticle. As an example, by exchanging negatively charged amino acids for hydrophobic amino acids, the negative surface potential in the region of the internal cavity can be reduced. In this manner, a significant increase in the adsorption capacity can be obtained for the PBUTs, which are also negatively charged under physiological conditions (FIG. 5a, Ftn^{(neg)}-Ap=Ftn^{(neg)}-Ap4, SEQ ID NO: 17). Because the modified region lies in the vicinity of the threefold channel, it can be assumed that the increased adsorption is due to an improved material transport into the internal region of the apoferritin nanoparticle. The material transport into the region of the internal cavity can in turn be improved by modifications in the region of the threefold channel. As an example, the exchange of amino acids with charged or sterically hindered residues for the amino acid alanine led to a significant increase in the adsorption capacity (FIG. 5a, Ftn^{(neg)}-Ap+channel=Ftn^{(neg)}-Ap4-3A, SEQ ID NO: 18). Binding sites created by amino acid exchange can be combined with modifications which improve material transport in order to further improve the adsorption. In addition, variants with more than one binding site per apoferritin subunit are possible.

In the variant Ftn^{(neg)}-Ap4 (SEQ ID NO: 17) that has already been mentioned above, amino acids with negatively charged side chains, such as glutamine and aspartic acid, were replaced at four positions (E61, E62, D131 and E140) per subunit in the internal void by nonpolar or aromatic derivatives (E61V, E62A, D131F and E140W). Because of the surface potential, there was a shift in the polarity from negative to uncharged (not shown). However, a negatively charged region remained on the internal surface, because the amino acids responsible for it are to some extent hidden in the protein backbone and mutations at these sites were left out in order to maintain the stability of the protein, Because the amino acids which are introduced carry hydrophobic or even aromatic side chains, it is possible for these residues to be capable of additionally functioning as adsorption sites for the partially hydrophobic PBUTs. In order to be able to distinguish whether possible effects on the adsorption capacities are due to a general reduction in the negative surface charge or to a specific adsorption of hydrophobic residues, a further variant with the designation Ftn^{(neg)}-Ap16 was developed (SEQ ID NO: 20). Like the Ap4 variant, the four charged amino acids at positions 61, 62, 131 and 140 were replaced by nonpolar derivatives (D at position 131 not by F, but by W) and twelve additional nonpolar amino acids were introduced at surface-exposed positions.

The adsorption capacities of Ftn^{(neg)}-Ap4 and Ftn^{(neg)}-Ap16 were determined with respect to Ftn^{(neg)} for both crystalline and also for non-crystalline forms (see FIG. 6; crystalline forms, left hand bar; non-crystalline forms, right hand bar). Upon comparison of the adsorption capacities of the crystalline materials in respect of indoxyl sulfate (IS), no significant difference between unmodified (Ftn^{(neg)} and modified ferritin (Ftn (neg)_Ap4, Ftn^{(neg)}-Ap16) was observed (see FIG. 6). For the non-crystalline material, however, in the case of both Ap variants, a significant increase in the adsorption capacity could be observed. Despite the 12 additional hydrophobic amino acids, the adsorption capacity of Ftn^{(neg)}-Ap16 is similar to that of Ftn^{(neg)}-Ap4. The reduction in the negative surface charge therefore appears to be the main reason for the increased PBUT adsorption. This agrees with the assumption that the introduction of hydrophobic molecules brings with it no significant improvement in the adsorption capacity, and the change in the adsorption capacity is ascribed to a general reduction in negative charges.

In addition to the reduction in the negative charge in the internal void and in the pores of the protein cage, a further strategy for increasing the adsorption of PBUTs was pursued. In order to stabilize both the polar as well as the hydrophobic portion of the PBUTs, the creation of a unique binding site by introducing suitable amino acids was envisaged. To this end, ligand docking protocols of the Rosetta software suite (see [40]) were used. IS was used as the ligand. Three further possible ferritin variants were determined, Ftn^{(neg)}-dock03 (SEQ ID NO: 21), Ftn^{(neg)}-dock23 (SEQ ID NO: 22) and Ftn^{(neg)}-dock43 (SEQ ID NO: 22). The binding sites were located in the vicinity of the threefold channel (Ftn^{(neg)}-dock03), in the center of the subunit (Ftn^{(neg)}-dock23) and in the vicinity of the fourfold channel (Ftn^{(neg)}-dock43).

The adsorption capacities of the new variants with respect to IS were determined for crystalline assemblies and are shown in FIG. 7. As can be seen in FIG. 7, the adsorption capacity for all three variants compared with unmodified Ftn^{(neg)} are significantly improved (Ftn^{(neg)}-03=Ftn^{(neg)}-dock03, Ftn^{(neg)}-23=Ftn^{(neg)}-dock23, Ftn^{(neg)}-43=Ftn^{(neg)}-dock43). At almost double the value, the variant Ftn^{(neg)}-03 exhibited the highest adsorption capacity.

The three variants Ftn^{(neg)}-dock03, Ftn^{(neg)}-dock23 and Ftn^{(neg)}-dock43 developed specifically for IS were also determined as regards their adsorption capacity in respect of pCS and PheAc, as can be seen in FIG. 8. Here, an interesting trend can be observed: while for IS, the variants Ftn^{(neg)}-03 and −43 both perform better than Ftn^{(neg)}-23, the variant Ftn^{(neg)}-23 performs better for pCS- and PheAc adsorption than the other variants. These results indicate that a kind of selectivity can be introduced into the binding sites in which unique features of the toxins are targeted.

Furthermore, a test was carried out as to whether the various features for increasing the binding capacity of the proteins could be combined in one structure. The mutations which form the binding site in Ftn^{(neg)}-43 were combined with the mutations of Ftn^{(neg)}-Ap4-3A in order to reduce the surface charge at the internal void and the threefold channel, whereupon the variant Ftn^{(neg)}-Ap4-3A-43 was produced.

The adsorption capacity of this variant was determined for the adsorbent with non-crystalline morphology and compared with the results for unmodified Ftn^{(neg)} and the aforementioned variants (FIG. 9). The measured values indicate that individual positive effects due to different modifications can be added together by combining the mutations into one structure. The variant Ftn^{(neg)}-Ap4-3A-43 exhibits the highest adsorption capacities of all of the variants investigated in this study.

The advantage with simply varying the properties of the apoferritin nanoparticle in accordance with the invention by amino acid exchange is that the proteins can be used without further chemical modification. This reduces the probability of allergic reactions or other side effects and makes it less expensive to produce than functionalized variants. Stronger binding sites can be designed. Furthermore, the present invention opens up the possibility of providing other apoferritin nanoparticles or apoferritin-nanoparticle compositions with further improved affinities, adapted to specific toxins as appropriate.

### REFERENCES

[1] Bikbov, B. et al., 2020, Global, regional, and national burden of chronic kidney disease, 1990-2017: a systematic analysis for the Global Burden of Disease Study 2017, The Lancet 395, 709-733
[2] Schlieper, G., Hess, K, Floege J., Marx, N., 2016, The vulnerable patient with chronic kidney disease, Nephrology Dialysis Transplantation 31, 382-390, doi.org/10.1093/ndt/gfv041
[3] Ito, S.; Yoshida, M. Protein-Bound Uremic Toxins: New Culprits of Cardiovascular Events in Chronic Kidney Disease Patients. Toxins 2014, 6, 665-678. doi.org/10.3390/toxins6020665
[4] Karbowska, M.; Kaminski, T. W.; Marcinczyk, N.; Misztal, T.; Rusak, T.; Smyk, L.; Pawlak, D. The Uremic Toxin Indoxyl Sulfate Accelerates Thrombotic Response after Vascular Injury in Animal Models. Toxins 2017, 9, 229. doi.org/10.3390/toxins9070229
[5] Dou L., Bertrand, E., Cerini, C., Faure, V., Sampol, J., Vanholder, R., Berland, Y, Brunet, P., 2004, The uremic solutes p-cresol and indoxyl sulfate inhibit endothelial proliferation and wound repair, Kidney International 65, 442-451, doi.org/10.1111/j.1523-1755.2004.00399.x
[6] Lekawanvijit, S., Kompa, A. R., Krum, H., 2016, Protein-bound uremic toxins: a long overlooked culprit in cardiorenal syndrome, Am J Physiol Renal Physiol 311: F52-F62, doi.org/10.1152/ajprenal.00348.2015
[7] Sun C Y, Chang S C, Wu M S (2012) Uremic Toxins Induce Kidney Fibrosis by Activating Intrarenal Renin-Angiotensin-Aldosterone System Associated Epithelial-to-Mesenchymal Transition. PLOS ONE 7 (3): e34026. doi.org/10.1371/journal.pone.0034026
[8] Yu M, Kim, YJ, Kang, D-H, 2011, Indoxyl Sulfate-Induced Endothelial Dysfunction in Patients with Chronic Kidney Disease via an Induction of Oxidative Stress, CJASN 6, 30-39; DOI: 10.2215/CJN.05340610
[9] Liabeuf, S., Barreto, D. V., Barreto, F. C., Meert, N., Glorieux, G., Schepers, E., Temmar, M., Choukroun, G., Vanholder, R., Massy, Z. A., on behalf of the European Uraemic Toxin Work Group (EUTox), 2010, Free p-cresylsulphate is a predictor of mortality in patients at different stages of chronic kidney disease, Nephrology Dialysis Transplantation 25, 1183-1191, doi.org/10.1093/ndt/gfp592
[10] Bammens, B., Evenepoel, P., Keuleers, H., Verbeke, K., Vanrenterghem Y., 2006, Free serum concentrations of the protein-bound retention solute p-cresol predict mortality in hemodialysis patients, Kidney International 69, 1081-1087, doi.org/10.1038/sj.ki.5000115
[11] Sirich, T. L., Fong, K., Larive, B., Beck, G. J., Chertow, G. M., Levin, N. W., Kliger, A. S., Plummer, N. S., Meyer, T. W., 2017, Limited reduction in uremic solute concentrations with increased dialysis frequency and time in the Frequent Hemodialysis Network Daily Trial, Kidney International 91, 1186-1192, doi.org/10.1016/j.kint.2016.11.002
[12] Vanholder, R., De Smet, R., Glorieux, G., Argilés, A., Baurmeister, U., Brunet, P., Clark, W., Cohen, G., De Deyn, P. P., Deppisch, R., Descamps-Latscha, B., Henle, T., Jörres, A. Lemke, H. D., Massy, Z. A., Passlick-Deetjen, J., Rodriguez, M., Stegmayr, B., Stenvinkel, P., Tetta, C., Wanner, C., Zidek, W., 2003, For the European Uremic Toxin Work Group (EUTox), Review on uremic toxins: Classification, concentration, and interindividual variability, Kidney International 63, 1934-1943, doi.org/10.1046/j.1523-1755.2003.00924.x
[13] Jansen, J., Jankowski, J., Gajjala, P. R., Wetzels, J. F. M., Masereeuw, R., 2017, Disposition and clinical implications of protein-bound uremic toxins, Clin Sci (Lond) 131, 1631-1647, doi.org/10.1042/CS20160191
[14] Itoh, Y., Ezawa, A., Kikuchi, K. et al. Protein-bound uremic toxins in hemodialysis patients measured by liquid chromatography/tandem mass spectrometry and their effects on endothelial ROS production. Anal Bioanal Chem 403, 1841-1850 (2012). doi.org/10.1007/s00216-012-5929-3
[15] Madero, M., Cano, K. B., Campos, I., Tao, X., Maheshwari, V., Brown, J., Cornejo, B., Handelman, G., Thijssen, S., Kotanko, P., 2019, Removal of Protein-Bound Uremic Toxins during Hemodialysis Using a Binding Competitor, CJASN 14, 394-402, doi: 10.2215/CJN.05240418
[16] Urquhart, B. L., Freeman, D. J., Spence, J. D., House, A. A., 2006, The Effect of Mesna on Plasma Total Homocysteinee Concentration in Hemodialysis Patients, American Journal of Kidney Diseases, Volume 49, Issue 1, 109-117, doi.org/10.1053/j.ajkd.2006.10.002
[17] Perna, A. F., Violetti, E., Lanza, D., Sepe, I., Bellinghieri, G., Savica, V., Santoro, D., Satta, E., Cirillo, G., Lupo A., Abaterusso, C., Raiola, I., Raiola, P., Coppola, S., Di Iorio, B., Tirino, G., Cirillo, M., Ingrosso, D., De Santo, N. G., 2012, Therapy of Hyperhomocysteineemia in Hemodialysis Patients: Effects of Folates and N-acetylcysteinee, Journal of Renal Nutrition 22, 507-514.el, doi.org/10.1053/j.jrn.2011.10.007
[18] Yamamoto, S., Kazama, J., Omori, K. et al. Continuous Reduction of Protein-Bound Uraemic Toxins with Improved Oxidative Stress by Using the Oral Charcoal Adsorbent AST-120 in Haemodialysis Patients. Sci Rep 5, 14381 (2015), doi.org/10.1038/srep14381
[19] Rocchetti, M. T.; Cosola, C.; di Bari, I.; Magnani, S.; Galleggiante, V.; Scandiffio, L.; Dalfino, G.; Netti, G. S.; Atti, M.; Corciulo, R.; Gesualdo, L. Efficacy of Divinylbenzenic Resin in Removing Indoxyl Sulfate and P-cresol Sulfate in Hemodialysis Patients: Results from an In Vitro Study and an In Vivo Pilot Trial (xuanro4-Nature 3.2). Toxins 2020, 12, 170. doi.org/10.3390/toxins12030170
[20] Saar-Kovrov, V, Zidek, W, Orth-Alampour, S, Fliser, D, Jankowski, V, Biessen, EAL, Jankowski, J (University hospital, Aachen, Germany; Maastricht University Medical Center, Maastricht, the Netherlands; Charité-Universitätsmedizin Berlin, Berlin, Germany; Saarland University Medical Center, Homburg, Germany). Reduction of protein-bound uraemic toxins in plasma of chronic renal failure patients: A systematic review (Review). J Intern Med 2021; 290:499-526.
[21] Brettschneider, F., Tö11e, M., von der Giet, M., Passlick-Deetjen, J., Steppan, S., Peter, M., Jankowski, V., Krause, A., Kühne, S., Zidek, W. and Jankowski, J. (2013), Thoughts and Progress. Artificial Organs, 37:409-416. doi.org/10.1111/j.1525-1594.2012.01570.x
[22] Falkenhagen, D., Strobl, W., Vogt, G., Schrefl, A., Linsberger, I., Gerner, F. J. and Schoenhofen, M. (1999), Fractionated Plasma Separation and Adsorption System: A Novel System for Blood Purification to Remove Albumin Bound Substances. Artificial Organs, 23:81-86, doi.org/10.1046/j.1525-1594.1999.06292.x
[23] Sternkopf, M.; Thoröe-Boveleth, S.; Beck, T.; Oleschko, K.; Erlenkötter, A.; Tschulena, U.; Steppan, S.; Speer, T.; Goettsch, C.; Jankowski, V.; Jankowski, J.; Noels, H.; The European Uremic Toxin Work Group-EUTox. A Bifunctional Adsorber Particle for the Removal of Hydrophobic Uremic Toxins from Whole Blood of Renal Failure Patients. Toxins 2019, 11, 389, doi.org/10.3390/toxins11070389
[24] Kato, S., Otake, K., Chen, H., Akpinar, I., Buru, C. T., Islamoglu, T., Snurr, R. Q., Farha, O. K., 2019, Zirconium-Based Metal-Organic Frameworks for the Removal of Protein-Bound Uremic Toxin from Human Serum Albumin, Journal of the American Chemical Society 141, 2568-2576, DOI: 10.1021/jacs.8b12525
[25] Berge-Lefranc, D., Vagner, C., Calaf, R., Denoyel, R., Brunet, P., Ghobarkar, H., Schaf, O., 2012, In vitro elimination of protein bound uremic toxin p-cresol by MFI-type zeolites. Microporous and Mesoporous Materials 153, 288-293, 10.1016/j.micromeso.2011.11.024.
[26] Barreto, F. C., Barreto, D. V., Liabeuf, S., Meert, N., Glorieux, G., Temmar, M., Choukroun, G., Vanholder, R., Massy, Z. A., on behalf of the European Uremic Toxin Work Group (EUTox), 2009, Serum Indoxyl Sulfate Is Associated with Vascular Disease and Mortality in Chronic Kidney Disease Patients, CJASN 4 (10) 1551-1558; DOI: 10.2215/CJN.03980609
[27] Rifai K, Ernst T, Kretschmer U, Bahr M J, Schneider A, Hafer C, Haller H, Manns M P, Fliser D. Prometheus-a new extracorporeal system for the treatment of liver failure. J Hepatol. 2003 December; 39 (6): 984-90. doi: 10.1016/s0168-8278 (03) 00468-9.
[28] Zhang, C.; Zhang, X.; Zhao, G. ferritin Nanocage: A Versatile Nanocarrier Utilized in the Field of Food, Nutrition, and Medicine. Nanomaterials 2020, 10, 1894. doi.org/10.3390/nano10091894
[29] Hempstead PD, Yewdall S J, Fernie A R, Lawson D M, Artymiuk P J, Rice D W, Ford G C, Harrison P M. Comparison of the three-dimensional structures of recombinant human H and horse L ferritins at high resolution. J Mol Biol. 1997 May 2; 268 (2): 424-48. doi: 10.1006/jmbi.1997.0970. PMID: 9159481.
[30] Wang W, Malcolm BA. Two-stage PCR protocol allowing introduction of multiple mutations, deletions and insertions using QuikChange Site-Directed Mutagenesis. Biotechniques. 1999 April; 26 (4): 680-2. doi: 10.2144/99264st03. PMID: 10343905
[31] Künzle, M., Eckert, T., Beck T., 2016, Binary Protein Crystals for the Assembly of Inorganic Nanoparticle Superlattices, J. Am. Chem. Soc. 138, 12731-12734, doi.org/10.1021/jacs.6b07260
[32] Rayment, I., 2002, Small-Scale Batch Crystallization of Proteins Revisited: An Underutilized Way to Grow Large Protein Crystals, Structure 10, 147-151, doi.org/10.1016/S0969-2126 (02) 00711-6
[33] Hida M, Aiba Y, Sawamura S, Suzuki N, Satoh T, Koga Y. Inhibition of the accumulation of uremic toxins in the blood and their precursors in the feces after oral administration of Lebenin, a lactic acid bacteria preparation, to uremic patients undergoing hemodialysis. Nephron. 1996; 74 (2): 349-55. doi: 10.1159/000189334. PMID: 8893154.
[34] Jankowski J., van der Giet M., Jankowski, V., Schmidt, S., Hemeier, M., Mahn, B., Giebing, G., Tö11e, M., Luftmann, H., Schlüter, H., Zidek, W., Tepel M., 2003, Increased plasma phenylacetic acid in patients with end-stage renal failure inhibits iNOS expression, J Clin Invest. 112 (2): 256-264, doi.org/10.1172/JCI15524
[35] Lu L., Yeow J. T. W. An adsorption study of indoxyl sulfate by zeolites and polyethersulfone-zeolite composite Mater 2017; 120:328-335. doi: 10.1016/j.matdes.2017.01.094
[36] Pavlenko, D., Giasafaki, D., Charalambopoulou, G. et al. Carbon Adsorbents With Dual Porosity for Efficient Removal of Uremic Toxins and Cytokines from Human Plasma. Sci Rep 7, 14914 (2017), doi.org/10.1038/s41598-017-15116-y
[37] van Gelder M K, Middel I R, Vernooij R W M, Bots M L, Verhaar M C, Masereeuw R, Grooteman M P, Nubé M J, van den Dorpel M A, Blankestijn P J, Rookmaaker M B, Gerritsen KGF, 2020, Protein-Bound Uremic Toxins in Hemodialysis Patients Relate to Residual Kidney Function, Are Not Influenced by Convective Transport, and Do Not Relate to Outcome, Toxins 12(4):234, doi: 10.3390/toxins12040234. PMID: 32272776;
[38] Kim, M., Rho, Y., Jin, K. S., Ahn, B., Jung, S., Kim, H., Ree, M., 2011, pH-Dependent Structures of ferritin and apoferritin in Solution: Disassembly and Reassembly, Biomacromolecules 2011, 12, 5, 1629-1640, doi.org/10.1021/bm200026v
[39] Liu, X., Jin, W., Theil, E. C., 2003, Opening protein pores with chaotropes enhances Fe reduction and chelation of Fe from the ferritin biomineral, PNAS 100, 3653-3658, doi.org/10.1073/pnas.0636928100
[40] Moretti, R., Bender, B. J., Allison, B., Meiler, J. (2016), Rosetta and the Design of Ligand Binding Sites, In: Stoddard, B. (eds) Computational Design of Ligand Binding Proteins, Methods in Molecular Biology 1414, pp. 47-62, Humana Press, New York, NY, doi: 10.1007/978-1-4939-3569-7_4

1. An apoferritin nanoparticle for use as a toxin binder in hemodialysis, wherein the apoferritin nanoparticle comprises apoferritin subunits which form an internal void when assembled into the apoferritin nanoparticle, and wherein the apoferritin nanoparticle does not contain any nanoparticles in its internal void.

2. The apoferritin nanoparticle for use as a toxin binder in hemodialysis as claimed in claim 1, wherein at least one of the apoferritin subunits in the region of the internal void and/or in the region of a threefold channel and/or in the region of a fourfold channel has an amino acid sequence which is modified with respect to the wild-type sequence.

3. The apoferritin nanoparticle for use as a toxin binder in hemodialysis as claimed in claim 2, wherein the at least one apoferritin subunit with an amino acid sequence which is modified with respect to the wild-type sequence in the region of the internal void has at least one additional cysteine residue or a cysteine residue in a different position in the sequence compared with the wild-type sequence, and wherein the at least one apoferritin subunit with an amino acid sequence which is modified with respect to the wild-type sequence is functionalized or can be functionalized by covalent binding of an organic compound residue to the cysteine residue.

4. The apoferritin nanoparticle for use as a toxin binder in hemodialysis as claimed in claim 3, wherein the at least one apoferritin subunit with an amino acid sequence which is modified with respect to the wild-type sequence has, compared with the wild-type sequence, two, three or four cysteine residues in the region of the internal void to which a respective organic compound residue is covalently bonded.

5. The apoferritin nanoparticle for use as a toxin binder in hemodialysis as claimed in claim 3, wherein the covalently bound organic compound residue disposed at the at least one additional cysteine residue or cysteine residue disposed in a different position in the sequence is selected from the group consisting of N-phenylacetamidyl, N-decylacetamidyl, N-(1-amino-2-phenylethyl) acetamidyl, N-(1-amino-2-phenylpropyl) acetamidyl, N-(1-amino-2-phenylbutyl) acetamidyl, N-(1,1-diamino-2-phenylethyl) acetamidyl, N-(1,1-diamino-2-phenylpropyl) acetamidyl, N-(1,1-diamino-2-phenylbutyl) acetamidyl, N-(3,4,5-tris(trifluoromethyl)phenyl) acetamidyl, N-(3,4,5-trinitrophenyl) acetamidyl, N-(1-amino-2-(3,4,5-trinitrophenyl)ethyl) acetamidyl and N-(2-(heptylamino)ethyl) acetamidyl.

6. The apoferritin nanoparticle for use as a toxin binder in hemodialysis as claimed in claim 1, wherein the amino acid sequence of the at least one apoferritin subunit with an amino acid sequence which is modified with respect to the wild-type sequence is modified compared with the wild-type sequence in the region of the void in a manner such that the apoferritin nanoparticle has a higher binding affinity for a toxin compared with an apoferritin nanoparticle produced from apoferritin subunits the amino acid sequence of which has not been modified with respect to the wild-type sequence.

7. The apoferritin nanoparticle for use as a toxin binder in hemodialysis as claimed in claim 1, wherein the amino acid sequence of the at least one apoferritin subunit with an amino acid sequence which is modified compared with the wild-type sequence is modified compared with the wild-type sequence in the region of a threefold channel in a manner such that the transport of a toxin into the void of the apoferritin nanoparticle is facilitated compared with an apoferritin nanoparticle with an unmodified amino acid sequence.

8. The apoferritin nanoparticle for use as a toxin binder in hemodialysis as claimed in claim 1, wherein the toxin is a protein-bound uremic toxin which is selected from the group consisting of indoxyl sulfate, para-cresyl sulfate, phenyl acetate and p-hydroxyhippuric acid.

9. The apoferritin nanoparticle for use as a toxin binder in hemodialysis as claimed in claim 1, wherein the apoferritin nanoparticle is composed of 24 apoferritin subunits, and at least two subunits have an amino acid sequence which is modified with respect to the wild-type sequence.

10. The apoferritin nanoparticle for use as a toxin binder in hemodialysis as claimed in claim 9, wherein the subunits have an amino acid sequence which is modified with respect to the wild-type sequence which is selected from one of the amino acid sequences in accordance with SEQ ID NO: 13-24.

11. A composition for use as a toxin binder in hemodialysis, comprising a plurality of apoferritin nanoparticles for use as a toxin binder in hemodialysis as claimed in claim 1.

12. The composition for use as a toxin binder in hemodialysis as claimed in claim 11, wherein the plurality of apoferritin nanoparticles for use as a toxin binder in hemodialysis are crosslinked among one another.

13. A sorbent cartridge having an interior, comprising in its interior
a plurality of apoferritin nanoparticles for use as a toxin binder in hemodialysis, wherein the apoferritin nanoparticles comprise apoferritin subunits which form an internal void when assembled into an apoferritin nanoparticle, and wherein the apoferritin nanoparticle does not contain any nanoparticles in its internal void, or
a composition comprising a plurality of apoferritin nanoparticles for use as a toxin binder in hemodialysis wherein the apoferritin nanoparticles comprise apoferritin subunits which form an internal void when assembled into the apoferritin nanoparticle, and wherein the apoferritin nanoparticle does not contain any nanoparticles in its internal void.

14. The sorbent cartridge as claimed in claim 11, wherein the plurality of apoferritin nanoparticles or the composition are contained in a first compartment inside the sorbent cartridge which is separated from a second compartment inside the sorbent cartridge by one or more membranes, wherein the membrane or the membranes are permeable to unbound toxin molecules and/or to proteins with toxin molecules bound thereto, but not permeable to the apoferritin nanoparticles.

15. A dialysis system, comprising a dialyzer and at least one sorbent cartridge as claimed in claim 13.

16. The dialysis system as claimed in claim 15, wherein the at least one sorbent cartridge is disposed a) upstream, b) downstream in the direction of flow of the blood to be dialyzed, or c) in an auxiliary circuit to the dialysis circuit.

17. The apoferritin nanoparticle for use as a toxin binder in hemodialysis as claimed in claim 1, wherein the apoferritin nanoparticle is composed of 24 apoferritin subunits, and wherein at least six subunits have an amino acid sequence which is modified with respect to the wild-type sequence.

18. The apoferritin nanoparticle for use as a toxin binder in hemodialysis as claimed in claim 1, wherein the apoferritin nanoparticle is composed of 24 apoferritin subunits, and wherein at least 20 subunits have an amino acid sequence which is modified with respect to the wild-type sequence.

19. The apoferritin nanoparticle for use as a toxin binder in hemodialysis as claimed in claim 1, wherein the apoferritin nanoparticle is composed of 24 apoferritin subunits, and wherein all 24 subunits have an amino acid sequence which is modified with respect to the wild-type sequence.

20. The apoferritin nanoparticle for use as a toxin binder in hemodialysis as claimed in claim 1, wherein the subunits have at least one amino acid sequence which is modified with respect to the wild-type sequence and which is selected from one of the amino acid sequences selected from the group consisting of SEQ ID NO: 17, SEQ ID NO: 18, SEQ ID NO: 23, and SEQ ID NO: 24.


SEQUENCE LISTING (24 sequences)

SEQ ID NO: 1 - protein, 182 aa, Homo sapiens
TTASTSQVRQNYHQDSEAAINRQINLELYASYVYLSMSYYFDRDDVALKNFAKYFLHQSHEEREHAEKLMKLQNQRGGRIFLQDIKKPDCDDWESGLNAMECALHLEKNVNQSLLELHKLATDKNDPHLCDFIETHYLNEQVKAIKELGDHVTNLRKMGAPESGLAEYLFDKHTLGDSDNES

SEQ ID NO: 2 - protein, 174 aa, Homo sapiens
SSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLKHD

SEQ ID NO: 3 - DNA, 40 bp, synthetic construct
  source 1..40: mol_type = other DNA
caagaacgatccgcatctggccgatttcatcgaaacccac

SEQ ID NO: 4 - DNA, 47 bp, synthetic construct
  source 1..47: mol_type = other DNA
tgttgcactgaagaactttgcgtgttactttctgcatcagtcccatg

SEQ ID NO: 5 - DNA, 45 bp, synthetic construct
  source 1..45: mol_type = other DNA
tttcatcagtttctcggcatgacagcgttcttcatgggactgatg

SEQ ID NO: 6 - DNA, 44 bp, synthetic construct
  source 1..44: mol_type = other DNA
atccccgagttctttgatcgcacagacctgttcgttcagatagt

SEQ ID NO: 7 - DNA, 43 bp, synthetic construct
  source 1..43: mol_type = other DNA
cacactttgggtgattgcgataatgaatcgtaactcgagcacc

SEQ ID NO: 8 - DNA, 21 bp, synthetic construct
  source 1..21: mol_type = other DNA
gcccaggcagtcagatcatct

SEQ ID NO: 9 - DNA, 22 bp, synthetic construct
  source 1..22: mol_type = other DNA
ttgagggtttgctacaacatgg

SEQ ID NO: 10 - DNA, 18 bp, synthetic construct
  source 1..18: mol_type = other DNA
caaccgcgagaagatgac

SEQ ID NO: 11 - DNA, 18 bp, synthetic construct
  source 1..18: mol_type = other DNA
gtccatcacgatgccagt

SEQ ID NO: 12 - protein, 182 aa, synthetic construct
TTASTSQVRQNYHQDSEEAINRQINLELYASYVYLSMSYYFDRDDVALKNFAKYFLHQSHEEREHAEKLMKLQNQRGGRIFLQDIQKPDEDDWESGLNAMEEALELEKNVNQSLLELHKLATDKNDPHLCDFIETHYLNEQVKAIKELGDHVTNLRKMGAPESGLAEYLFDKHTLGDSDNES

SEQ ID NO: 13 - protein, 182 aa, synthetic construct
TTASTSQVRQNYHQDSEEAINRQINLELYASYVYLSMSYYFDRDDVALKNFACYFLHQSHEERCHAEKLMKLQNQRGGRIFLQDIQKPDEDDWESGLNAMEEALELEKNVNQSLLELHKLATDKNDPHLADFIETHYLNEQVCAIKELGDHVTNLRKMGAPESGLAEYLFDKHTLGDSDNES

SEQ ID NO: 14 - protein, 182 aa, synthetic construct
TTASTSQVRQNYHQDSEEAINRQINLELYASYVYLSMSYYFDRDDVALKNFACYFLHQSHEERCHAEKLMKLQNQRGGRIFLQDIQKPDEDDWESGLNAMEEALELEKNVNQSLLELHKLATDKNDPHLADFIETHYLNEQVCAIKELGDHVTNLRKMGAPESGLAEYLFDKHTLGDCDNES

SEQ ID NO: 15 - protein, 182 aa, synthetic construct
TTASTSQVRQNYHQDSEEAINRQINLELYASYVYLSMSYYFDRDDVALKNFACYFLHQSHEEREHAEKLMKLQNQRGGRIFLQDIQKPDEDDWESGLNAMEEALELEKNVNQSLLELHKLATDKNDPHLADFIETHYLNEQVKAIKELGDHVTNLRKMGAPESGLAEYLFDKHTLGDSDNES

SEQ ID NO: 16 - protein, 182 aa, synthetic construct
TTASTSQVRQNYHQDSEEAINRQINLELYASYVYLSMSYYFDRDDVALKNFACYFLHQSHEERCHAEKLMKLQNQRGGRIFLQDIQKPDEDDWESGLNAMEEALELEKNVNQSLLELHKLATDKNDPHLADFIETHYLNEQVKAIKELGDHVTNLRKMGAPESGLAEYLFDKHTLGDSDNES

SEQ ID NO: 17 - protein, 182 aa, synthetic construct
TTASTSQVRQNYHQDSEEAINRQINLELYASYVYLSMSYYFDRDDVALKNFAKYFLHQSHVAREHAEKLMKLQNQRGGRIFLQDIQKPDEDDWESGLNAMEEALELEKNVNQSLLELHKLATDKNDPHLCFFIETHYLNWQVKAIKELGDHVTNLRKMGAPESGLAEYLFDKHTLGDSDNES

SEQ ID NO: 18 - protein, 182 aa, synthetic construct
TTASTSQVRQNYHQDSEEAINRQINLELYASYVYLSMSYYFDRDDVALKNFAKYFLHQSHVAREHAEKLMKLQNQRGGRIFLQDIQKPDEDDWESGLNAMEEALELEKNVNQSLLELHKLAAAKADPHLCFFIETHYLNWQVKAIKELGDHVTNLRKMGAPESGLAEYLFDKHTLGDSDNES

SEQ ID NO: 19 - protein, 182 aa, synthetic construct
TTASTSQVRQNYHQDSEEAINRQINLELYASYVYLSMSYYFDRDDVALKNFAKYFLHQSHVAREHAEKLMKLQNQRGGRIFLQDIQKPDEDDWESGLNAMEEALELEKNVNQSLLELHKLATDKNDPFLCWFIETHYLVWQVVAIKELGDHVTNLRKMGAPESGLAEYLFDKHTLGDSDNES

SEQ ID NO: 20 - protein, 182 aa, synthetic construct
TTASTSQVRQNYHQDSEEAINRQINLELYASYVYLSMSYYFDRDDVALVNFAKFFLWLSHVARELAEKLMKLQNQRGGRIFLQDIQKPDEDDWESGLNAMEEALELEKNVNQSLLELHKLATDKNDPFLCWFIETYYLVWQVVAIKLLGDYVTALRKMGAPESGLAEYLFDKHTLGDSDNES

SEQ ID NO: 21 - protein, 182 aa, synthetic construct
TTASTSQVRQNYHQDSEEAINRQINLELYASYVYLSMSYYFDRDDVALKNFAKYFLHQSHEERRKAEELMRLQNDKGGRIFLQDIQKPDEDDWESGLNAMEEALELEKNVNQSLLELHKLATDKNDPELCEHIEKRHLRRQVKAIKELGDHVTNLRKMGAPESGLAEYLFDKHTLGDSDNES

SEQ ID NO: 22 - protein, 182 aa, synthetic construct
TTASTSQVRQNYHQDSEEAINRQINLELYASYVYLSMSYYFDRDDVALKNFAKYFLERSIGERGHAEDLMKLQNQRGGRIFLQDIQKPDEDDWESGLNAMEEALELEKNVNQSLLELHKLATDKNDPHLCDFIEDKHLEKQVKNIKELGDHVTNLRKMGAPESGLAEYLFDKHTLGDSDNES

SEQ ID NO: 23 - protein, 182 aa, synthetic construct
TTASTSQVRQNYHQDSEEAINRQINLELYASYVYLSMSYYFDRDDVALKNFAKYFLERSHEERRKAEELMKLQNQRGGRIFLQDIQKPDEDDWESGLNAMEEALELEKNVNQSLLELHKLATDKNDPHLCDFIEDRHLRRQVKAIKYLGDHVTNLRKMGAPESGLAEYLFDKHTLGDSDNES

SEQ ID NO: 24 - protein, 182 aa, synthetic construct
TTASTSQVRQNYHQDSEEAINRQINLELYASYVYLSMSYYFDRDDVALKNFAKYFLERSHVARRKAEELMKLQNQRGGRIFLQDIQKPDEDDWESGLNAMEEALELEKNVNQSLLELHKLAAAKADPHLCFFIEDRHLRRQVKAIKYLGDHVTNLRKMGAPESGLAEYLFDKHTLGDSDNES